(19) United States
(12) Patent Application Publication
(10) Pub. No.: US 2026/0118274 A1
(43) Pub. Date: Apr. 30, 2026

(54) SYSTEM AND METHOD FOR MONITORING CROPS

(71) Applicant: The United States of America, as Represented by the Secretary of Agriculture, Washington, DC (US)

(72) Inventor: CHRISTINE Y. CHANG, COLLEGE PARK, MD (US)

(21) Appl. No.: 18/931,591

(22) Filed: Oct. 30, 2024

(51) Int. Cl. G01N 21/84; A01G 7/04; G01D 5/14; G01J 1/42; G01N 21/31; G01N 21/64; G01N 33/00

(52) U.S. Cl. CPC G01N 21/84; A01G 7/045; G01D 5/145; G01J 1/4204; G01N 21/31; G01N 21/6486; G01N 33/0098; G01N 2021/8466

(57) ABSTRACT

Systems and methods for monitoring crops are disclosed. This disclosure relates to a system for monitoring crops, the system comprising a first support frame, the support frame comprising a first sensor rack coupled to the first support frame, the first sensor rack comprising a first sensor module; a movement module, the movement module comprising a first sensor rack motor, wherein the first sensor rack motor is configured to move the first sensor rack along the first support frame from a first starting location to a first ending location; and a hyperspectral module, wherein the first sensor module is coupled to the hyperspectral module.

### FIELD

The disclosed subject matter relates generally to systems and methods for monitoring crops. Specifically, the subject matter described herein relates to systems and methods to automatically track plant stress responses using hyperspectral reflectance, nighttime chlorophyll fluorescence, and imaging.

### BACKGROUND

Plant phenotyping is considered one of the bottlenecks of current agricultural research (Fiorani and Schurr 2013; Araus and Cairns 2014). Plant phenotyping is a scientific field that measures and analyzes plant traits to improve crop productivity and sustainability. It is the process of measuring a plant's structural and functional properties, such as growth, yield, and stress adaptation. Plant phenotyping refers to a quantitative description of the plant's anatomical, ontogenetical, physiological and biochemical properties. It uses a variety of measurements including destructive sample analyses and non-invasive technologies to track traits over time and screen genotypes.

Crop breeding approaches need to be improved to keep pace with the increasing demand on major grain crops (Tester and Langridge 2010) and other food commodities. The ability to monitor crop health and development efficiently and accurately is paramount for advancing agricultural research and optimizing crop production. Traditional phenotyping methods often involve labor-intensive and time-consuming processes, which limit their application to dynamic environments. Recent technological advancements have paved the way for the development of automated and high-throughput systems that significantly enhance phenotyping capabilities. High throughput phenotyping (HTP) has emerged as a critical tool in this regard, providing detailed insights into plant responses to various environmental stresses. Automated approaches for remote sensing-based quantification of several traits to monitor the plant stress response have been established throughout the last decade (Chen et al. 2014; Junker et al. 2015; Jin et al. 2023). Among these traits are plant architecture, height, growth, photosynthesis, biomass, chlorophyll status, senescence and vigor. These traits can be extracted from red, green, and blue (RGB) images, or they can be related to spectral parameters associated with plant traits derived from canopy reflectance, such as the normalized difference vegetation index (NDVI), to chlorophyll fluorescence, or to canopy temperature quantified via thermography (Araus and Cairns 2018).

Several high-throughput phenotyping systems have been developed to address the limitations of traditional methods (Jin et al. 2022). Some systems have introduced automated imaging technologies, including RGB, thermal, and hyperspectral imaging, to monitor various plant traits that allow for an assessment of crop traits at a sufficient throughput (Hairmansis et al. 2014; Asaari et al. 2019; Beauchêne et al. 2019). These systems, primarily designed for large-scale facilities, have significantly advanced the field by providing high-resolution data for phenotypic analysis. However, they often require extensive infrastructure and are primarily suitable for field environments, limiting their flexibility and applicability in diverse experimental conditions under controlled environments. Additionally, these systems typically focus on daytime measurements, neglecting the critical physiological processes that occur during the night to detect the stress response.

Chlorophyll fluorescence (ChlF) is an optical signal emitted from illuminated chlorophyll molecules and represents the fraction of absorbed light not used in photochemistry or dissipated by nonphotochemical quenching (Porcar-Castell et al., 2014). Sun-induced chlorophyll fluorescence (SIF), measured under natural sunlight, has been identified as a reliable indicator of photosynthesis and gross primary production (GPP) across various environmental conditions (Mohammed et al., 2019; Sun et al., 2017). Understanding the mechanistic aspects of the ChlF signal is crucial to detect plant stress, as it contains inherent information on both dynamic and sustained plant processes. Remote sensing techniques focused on the far-red and red spectral regions have proven effective in retrieving SIF signals from satellites and ground-based hyperspectral sensors (Frankenberg et al., 2011; Guanter et al., 2012). A new approach has recently emerged to measure nighttime ChlF spectra of plant canopies induced by light-emitting diodes (LEDs) (Romero et al., 2018, 2021). Blue LEDs (emitting light around 450-490 nanometers (nm)) are particularly effective, as ChlF is emitted in the red to far-red spectral range (650-800 nm), thus providing a pure chlorophyll fluorescence signal that is free from contamination from the actinic light source (Atherton et al., 2019). Its key advantage lies in its ability to enable dark-acclimated measurements of canopy-scale chlorophyll fluorescence, offering insights into vegetation responses under sustained environmental conditions, which helps assess plant stress impacts on fluorescence and potentially on leaf photosynthetic capacity (Rajewicz et al., 2023). Hyperspectral sensors can be used to retrieve the full emission spectrum of chlorophyll fluorescence. These spectra enable the evaluation of spectral variance components sensitive to chlorophyll content and the ratio of nonphotochemical quenching to photochemistry (Magney et al., 2019), along with calculating red to far-red fluorescence ratios, which are indicators of plant stress or changes in chlorophyll concentration (Bowling, et al., 2019; Ortiz-Bustos et al., 2016). Recently, two systems have been developed to track nighttime ChlF in plant canopies (Brissette et al. 2023; Wong et al. 2024). However, both systems are stationary, and thus limited to only phenotype one or a few plants.

While the existing systems and methods are useful to a degree, they still suffer from certain limitations. Therefore, there exists a need in the art for improved systems and methods for monitoring crops that solve or at least alleviate some or all of these problems.

### SUMMARY OF THE EMBODIMENTS

Systems and methods for monitoring crops are disclosed and claimed herein.

As described more fully below, the devices and processes of the embodiments disclosed permit improved systems and methods for monitoring crops. Further aspects, objects, desirable features, and advantages of the apparatus, systems, and methods disclosed herein will be better understood and apparent to one skilled in the relevant art in view of the detailed description and drawings that follow, in which various embodiments are illustrated by way of example. It is to be expressly understood, however, that the drawings are for the purpose of illustration only and are not intended as a definition of the limits of the claimed embodiments.

To this end, a crop monitoring system is provided, the system comprising: a first support frame, the support frame comprising a first sensor rack coupled to the first support frame, the first sensor rack comprising a first sensor module; a movement module, the movement module comprising a first sensor rack motor, wherein the first sensor rack motor is configured to move the first sensor rack along the first support frame from a first starting location to a first ending location; and a hyperspectral module, wherein the first sensor module is coupled to the hyperspectral module.

In some embodiments, the hyperspectral module comprises a high-resolution spectrometer configured to a spectral range of about 500-900 nm; a multiplexer coupled to the spectrometer; and at least one armored fiber optic cable coupled to the optical multiplexer. In various embodiments, the hyperspectral module comprises a spectrometer configured to a spectral range of about 500-900 nm and is configured to collect a hyperspectral reflectance from a subject crop. In some embodiments, the hyperspectral module comprises a high-resolution spectrometer configured to a spectral range of about 500-900 nm with a spectral resolution of about 0.6 nm full width at half maximum (FWHM). In some embodiments, the hyperspectral module comprises a high-resolution spectrometer; a multiplexer coupled to the spectrometer; and at least two armored fiber optic cables coupled to the optical multiplexer. In other embodiments, other spectral ranges and other spectral resolutions may be used. In various embodiments, the hyperspectral module comprises a multiplexer that aligns the spectrometer's optical inlet with one of a plurality of light-collecting channels, each channel equipped with an individual fiber optic for user configurability, or a dark measurement from a built-in dark channel. In some embodiments, the hyperspectral module comprises a multiplexer having a piezoelectric motor that aligns the spectrometer's optical inlet with one of a plurality of light-collecting channels, each channel equipped with an individual fiber optic for user configurability, or a dark measurement from a built-in dark channel. In certain embodiments, the hyperspectral module may comprise at least one armored fiber optic cable with about 200 μm core diameter connected to the optical multiplexer. In other embodiments, the hyperspectral module may comprise at least two armored fiber optic cables with about 200 μm core diameter connected to the optical multiplexer. In some embodiments, the fiber optic cables are coupled to the spectrometer. In certain embodiments, the fiber optic cables are directly coupled to the spectrometer.

In various embodiments, the first sensor rack may comprise a first sensor module having a lighting module for inducing a ChlF emission and a hyperspectral module for recording a ChlF signal. In certain embodiments, the first sensor rack motor is configured to move the first sensor rack along the first support frame from a first starting location to a first ending location, with stopping points determined by Hall effect sensors.

In some embodiments, the system further comprises a second sensor rack coupled to a second support frame, the second sensor rack comprising a second sensor module. In certain embodiments, the movement module further comprises a second sensor rack motor, the second sensor rack motor configured to move the second sensor rack along the second support frame from a second starting location to a second ending location.

In some embodiments, the system further comprises a second sensor rack coupled to a second support frame, the second sensor rack comprising a second sensor module comprising a hyperspectral module for recording canopy reflectance and an RGB imaging module for recording above-canopy images. In certain embodiments, the movement module further comprises a second sensor rack motor, the second sensor rack motor configured to move the second sensor rack along the second support frame from a second starting location to a second ending location, with stopping points determined by Hall effect sensors.

In various embodiments, the system further comprises a controller in electrical communication with the movement module, the controller configured to control the movement of the first sensor rack and the second sensor rack.

In some embodiments, the system further comprises an environmental module, the environmental module configured to collect a light intensity; and wherein the controller is in electrical communication with the environmental module to determine a daytime cycle lighting condition or a nighttime cycle lighting condition based on the light intensity from the environmental module. In various embodiments, the environmental module comprises a photosynthetically active radiation (PAR) sensor, which is used to detect the daytime or nighttime activity of the crop monitoring system. In certain embodiments, the environmental module comprises at least one soil time-domain-reflectometry (TDR) probe to monitor soil moisture and temperature and demonstrate system function. In various embodiments, the environmental module includes a datalogger which can connect to various environmental sensors based on needs determined by the user.

In certain embodiments, the crop monitoring system is an autonomous crop monitoring system. In an embodiment where the crop monitoring system is autonomous, the crop monitoring system may operate on its own without direct human intervention. In various embodiments, a user may input desired criteria into a user interface where those instructions are given to a controller, then after receiving the set of instructions from the user, the system may operate on by itself without further human intervention.

In some embodiments, the first sensor module includes one or more fiber optics from the hyperspectral module, configured to collect ChlF from a subject crop. In some embodiments, the first sensor module further comprises a light source, wherein the light source is configured to induce ChlF in a subject crop. In various embodiments, the first sensor module comprises a light box, where the light box may comprise the light source. In some embodiments, the light box may have at least one light. In some embodiments, the light source may be an LED. In certain embodiments the light source may be a blue light. In some embodiments the light source may be a blue LED light. In various embodiments, the light source may be a light having a wavelength of 380-500 nm. In some embodiments, the light source may be a light having a wavelength of 450-495 nm.

In certain embodiments, the second sensor module includes one or more fiber optics from the hyperspectral module, configured to collect hyperspectral reflectance from a subject crop. In some embodiments, the second sensor module further comprises an imaging module, wherein the imaging module comprises a camera, wherein the camera is configured to collect above-canopy RGB imagery of a subject crop. In some embodiments, the hyperspectral module is configured to collect a hyperspectral reflectance from a subject crop.

In various embodiments, the system further comprises a Hall effect sensor, where the Hall effect sensor is coupled to the support frame. In some embodiments, the system further comprises a magnet coupled to the first sensor rack, wherein the Hall effect sensor is configured to sense the magnet and send a signal to the controller to stop the movement of the first sensor rack such that the first sensor rack is over a subject crop. In certain embodiments, the Hall effect sensor is configured to sense the magnet and send a signal to the controller to stop the movement of the first sensor rack such that the first sensor rack is over a row of subject crops aligned with the Hall effect sensor.

In some embodiments, the first sensor rack is located below the second rack, such that the first rack is configured to travel parallel to the second sensor rack between a subject crop and the second sensor rack. In some embodiments, the first sensor module comprises a fiber optic sensor.

In certain embodiments, the system further comprises a first drive shaft, wherein the first drive shaft is operatively connected to the first sensor rack motor; a first drive chain operatively connected to the first drive shaft and a first front top axle; and a first rack chain operatively connected to the first front top axle and a first front bottom axle at a front end of the first support frame, the first rack chain operatively connected to the first sensor rack.

In one form, the present disclosure provides an automated crop monitoring system, the system comprising: a control module, configured to direct operations of the system; a first support frame; a first sensor rack coupled to the first support frame, the first sensor rack comprising at least one sensor coupled to the first sensor rack; a movement module, the movement module comprising a first sensor rack motor, wherein the first sensor rack motor is configured to move the first sensor rack along the first support frame from a first starting location to a first ending location; at least one Hall effect sensor coupled to first support frame; and a magnet coupled to the first sensor rack, wherein the at least one Hall effect sensor is configured to sense the magnet and send a signal to the control module to stop the movement of the first sensor rack such that the first sensor rack is over a subject crop.

In some embodiments, the system further comprises a hyperspectral module, wherein the first sensor module is coupled to the hyperspectral module. In certain embodiments, the system further comprises a second sensor rack coupled to a second support frame, the second sensor rack comprising a second sensor module. In some embodiments, the movement module further comprises a second sensor rack motor, the second sensor rack motor configured to move the second sensor rack along the second support frame from a second starting location to a second ending location.

In various embodiments, the system further comprises an environmental module, the environmental module configured to collect a light intensity; and wherein the controller is in electrical communication with the environmental module to determine a daytime cycle lighting condition or a nighttime cycle lighting condition based on the light intensity from the environmental module. In some embodiments, the first sensor module further comprises a light source, wherein the light source is configured to induce a chlorophyll fluorescence in a subject crop. In certain embodiments, the system further comprises an imaging module, wherein the imaging module comprises a camera, wherein the camera is configured to collect above-canopy RGB imagery of the subject crop. In some embodiments, the system further comprises a first drive shaft, wherein the first drive shaft is operatively connected to the first sensor rack motor. In certain embodiments, the system further comprises a light module, wherein the light module comprises a light source, and wherein the light source is configured to induce a chlorophyll fluorescence in a subject crop.

In one form, the present disclosure provides a method for monitoring crop performance, the method comprising the steps of: determining a nighttime cycle; moving and aligning a first sensor rack to a configuration to sense and record data from a subject crop during the nighttime cycle; determining a daytime cycle; and moving and aligning a second sensor rack to a configuration to sense and record data from the subject crop during the daytime cycle.

In various embodiments, the method further comprises the step of scanning a white reference at a home position of the second sensor rack after determining the daytime cycle. In certain embodiments, the method further comprises the steps of capturing an image at a first sensor position and transferring the image to a main controller; and taking a measurement at the first sensor position by a hyperspectral sensor.

In some embodiments, the method further comprises the step of calculating a radiance using a radiometric calibration file; calculating a raw reflectance; calculating a corrected reflectance; saving at least one of a raw spectral data, a radiance, a raw reflectance, and a corrected reflectance; moving the second sensor rack to a home position; and taking at least one environmental measurement. In various embodiments, the method further comprises the step of turning on a blue light at a first sensor position; taking an image of a subject crop at the first sensor position; transferring the image to a controller; and taking a measurement at the first sensor position. In certain embodiments, the method further comprises the step of calculating a fluorescence using a radiometric calibration file; saving at least one of a raw spectral data and a fluorescence; moving the first sensor rack to a home position; and taking at least one environmental measurement.

In one form, the present disclosure provides a method for monitoring crop performance, the method comprising the steps of: automatically determining a nighttime cycle; automatically moving and aligning a first sensor rack to a configuration to sense and record data from a subject crop during the nighttime cycle; automatically determining a daytime cycle; and automatically moving and aligning a second sensor rack to a configuration to sense and record data from the subject crop during the daytime cycle. In various embodiments, the method further comprises the step of scanning a white reference at a home position of the second sensor rack after automatically determining the daytime cycle.

In one form, the present disclosure provides an autonomous crop monitoring system, the system comprising: a first sensor rack, the first sensor rack comprising a first sensor module, the first sensor module comprising a first hyperspectral sensor, and a light module; a second sensor rack, the second sensor rack comprising a second sensor module, an imaging sensor and a second hyperspectral sensor; a hyperspectral module, the hyperspectral module coupled to the first hyperspectral sensor and the second hyperspectral sensor; a movement module, the movement module comprising a first sensor rack motor, the first sensor rack motor configured to move the first rack from a starting location to an ending location; the movement module comprising a second sensor rack motor, the second sensor rack motor configured to move the second rack from a starting location to an ending location; a controller in electrical communication with the movement module, the controller configured to: control the movement of the first rack and the second rack; determine a daytime cycle lighting condition or a nighttime cycle lighting condition based on inputs from the environmental module.

In one form, the present disclosure provides a crop monitoring system, the system comprising: a first sensor rack, the first sensor rack comprising a first sensor module, the first sensor module comprising a first hyperspectral sensor; and a light module; a second sensor rack, the second sensor rack comprising a second sensor module, an imaging sensor and a second hyperspectral sensor; a hyperspectral module, the hyperspectral module coupled to the first hyperspectral sensor and the second hyperspectral sensor; a movement module, the movement module comprising a first sensor rack motor, the first sensor rack motor configured to move the first rack from a starting location to an ending location; the movement module comprising a second sensor rack motor, the second sensor rack motor configured to move the second rack from a starting location to an ending location; a Hall effect sensor, the Hall effect sensor coupled to a rail of a frame; a magnet coupled to the first rack, such that when the Hall effect sensor senses the magnet the Hall effect sensor sends a signal to the controller, the controller then stops the movement of the first rack such that the first rack is over a target location.

In some embodiments, the system further comprises a controller in electrical communication with the movement module, the controller configured to: control the movement of the first rack and the second rack; determine a daytime cycle lighting condition or a nighttime cycle lighting condition based on inputs from the environmental module; and receive sensor inputs.

In one form, the present disclosure provides an automated high-throughput phenotyping (HTP) system for monitoring crop performance, comprising: a control module, configured to direct operations of the system; a light module, configured to activate a light to induce a chlorophyll fluorescence; and a hyperspectral module, configured to collect a hyperspectral reflectance; an imaging module, configured to collect above-canopy RGB imagery; and an environmental module, configured to collect light intensity to determine a day or night status.

In various embodiments, the system further comprises a movement module, the movement module comprising a first sensor rack motor, the first sensor rack motor operatively connected to a first drive shaft, a first drive chain operatively connected to the first drive shaft and a first front top axle, a first rack chain operatively connected to the first front top axle and a first front bottom axle at a front end of a frame, the first rack chain operatively connected to a first sensor rack, the first rack chain operatively connected to a first rear top axle and a first rear bottom axle at a rear end of the frame.

In certain embodiments, the movement module comprises a second sensor rack motor, the second sensor rack motor operatively connected to a second drive shaft, a second drive chain operatively connected to the second drive shaft and a second front top axle, a second rack chain operatively connected to the second front top axle and a second front bottom axle at a front end of a frame, the second rack chain operatively connected to a second sensor rack, the second rack chain operatively connected to a second rear top axle and a second rear bottom axle at a rear end of the frame.

In some embodiments, the system further comprises where the control module is further configured to provide a user interface and manage data; the hyperspectral module is further configured to collect ChlF emission spectra; the light module is further configured to activate the blue light during nighttime to induce ChlF in a subject crop for measurements; and the environmental module is further configured to collect light intensity to determine day or night status, and wherein the environmental module further comprises environmental sensors to detect at least one of a soil water availability, a temperature, and a carbon dioxide level.

In one form, the present disclosure provides an automated high-throughput phenotyping system for monitoring crop performance, comprising: a control module, configured to direct operations of the system; a first frame comprising a first rail of the first frame and a second rail of the first frame; a first sensor rack, comprising at least one sensor coupled to the first sensor rack; at least one Hall effect sensor coupled to the first rail of the first frame; at least one magnet coupled to a first end of the first sensor rack, such that the magnet is configured to trigger the Hall effect sensors to stop the first sensor rack.

In one form, the present disclosure provides an autonomous crop monitoring system, the system comprising: a bottom rack, the bottom rack comprising at least one bottom rack sensor module, the at least one bottom rack sensor module comprising a bottom rack hyperspectral sensor and a light module; a top rack, the top rack comprising a at least one top sensor module, an imaging sensor and a top rack hyperspectral sensor; a hyperspectral module, the hyperspectral module coupled to the bottom rack hyperspectral sensor and the top rack hyperspectral sensor; a movement module, the movement module comprising a bottom rack motor, the bottom rack motor configured to move the bottom rack from a starting location to an ending location; the movement module comprising a top rack motor, the top rack motor configured to move the top rack from a starting location to an ending location; and a controller in electrical communication with the movement module, the controller configured to control the movement of the bottom rack and the top rack, and receive sensor inputs.

These and other objects, features, aspects, and advantages of the present patent document will become better understood with reference to the following description and accompanying drawings.

### BRIEF DESCRIPTION OF THE DRAWINGS

FIG. 1 illustrates a schematic diagram of a perspective view of an embodiment of a system for monitoring crops of the present patent document.

FIG. 2 illustrates a schematic diagram of a top view of an embodiment of a system for monitoring crops of the present patent document shown in FIG. 1.

FIG. 3 illustrates a schematic diagram of a side view of a front gear system of the system for monitoring crops of the present patent document shown in FIG. 1.

FIG. 4 illustrates a schematic diagram of a side view of a rear gear system of the system for monitoring crops of the present patent document shown in FIG. 1.

FIG. 5 illustrates a schematic diagram of a perspective view of a white reference system of the system for monitoring crops of the present patent document shown in FIG. 1.

FIG. 6 illustrates a schematic diagram of a side view of a Hall effect system of the system for monitoring crops of the present patent document shown in FIG. 1.

FIG. 7 illustrates a schematic diagram of a bottom view of a blue light system of the system for monitoring crops of the present patent document shown in FIG. 1.

FIG. 8 illustrates a process for monitoring crops of the present patent document.

FIG. 9 illustrates a schematic diagram of some modules of the system for monitoring crops of the present patent document.

FIG. 10 illustrates another schematic diagram of the system for monitoring crops of the present patent document.

FIG. 11A illustrates a graph of a light condition in a growth chamber and FIG. 11B illustrates a graph of example correction factors of the system in a growth chamber with non-uniformly distributed lighting for monitoring crops of the present patent document.

FIG. 12A illustrates a graph of motor path error positions versus chamber positions and FIG. 12B illustrates a graph of sensor bar error positions versus chamber positions of the system for monitoring crops of the present patent document.

FIGS. 13A, 13B, and 13C illustrate graphs of conditions of the target plants during an experiment.

FIGS. 14A and 14B illustrate graphs of blue light induced chlorophyll fluorescence patterns, and FIG. 14C illustrates red light to far-red light ration (R:FR Ratio) in various conditions during an experiment.

FIG. 15 illustrates Pearson correlations among reflectance-based vegetation indices derived from the system for monitoring crops of the present patent document (PhenoGazer, PG) and a commercial system (TraitFinder, TF).

FIG. 16 illustrates a schematic diagram of a power wiring configuration of the system for monitoring crops of the present patent document.

FIG. 17 illustrates a schematic diagram of a Hall effect wiring configuration of the system for monitoring crops of the present patent document.

FIG. 18 illustrates a schematic diagram of communications from a main controller of the system for monitoring crops of the present patent document.

FIG. 19 illustrates a schematic diagram of an imaging module of the system for monitoring crops of the present patent document.

FIG. 20 illustrates a schematic diagram of a controller for the lighting module of the system for monitoring crops of the present patent document.

FIG. 21 illustrates a schematic diagram of an LED controller relay of the system for monitoring crops of the present patent document.

FIG. 22 illustrates a schematic diagram of a motor interrupt relay of the system for monitoring crops of the present patent document.

Note that assemblies/systems in some of the figures may contain multiple examples of essentially the same component. For simplicity and clarity, only a small number of the example components may be identified with a reference number. Unless otherwise specified, other non-referenced components with essentially the same structure as the exemplary component should be considered to be identified by the same reference number as the exemplary component. Further, unless specifically indicated otherwise, drawing components may or may not be shown to scale.

### DETAILED DESCRIPTION OF PREFERRED EMBODIMENTS

Reference will now be made to the drawings in which the various elements of the present disclosure will be given numerical designations and in which the present disclosure will be discussed so as to enable one skilled in the art to make and use the present disclosure. It is to be understood that the following description is only exemplary of the principles of the present disclosure, and should not be viewed as narrowing the claims. Additionally, it should be appreciated that the components of the individual embodiments discussed may be selectively combined in accordance with the teachings of the present disclosure. Furthermore, it should be appreciated that various embodiments will accomplish different objects of the present disclosure, and that some embodiments falling within the scope of the present disclosure may not accomplish all of the advantages or objects which other embodiments may achieve.

In accordance with the present disclosure, improved systems and methods for monitoring crops are disclosed which address, or at least ameliorate one or more of the problems of existing designs.

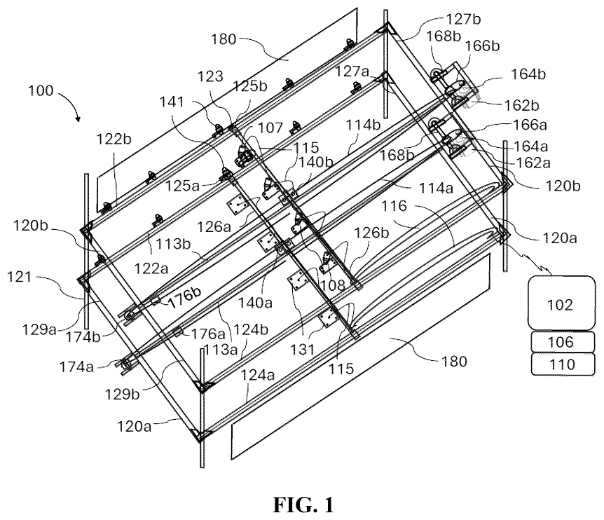

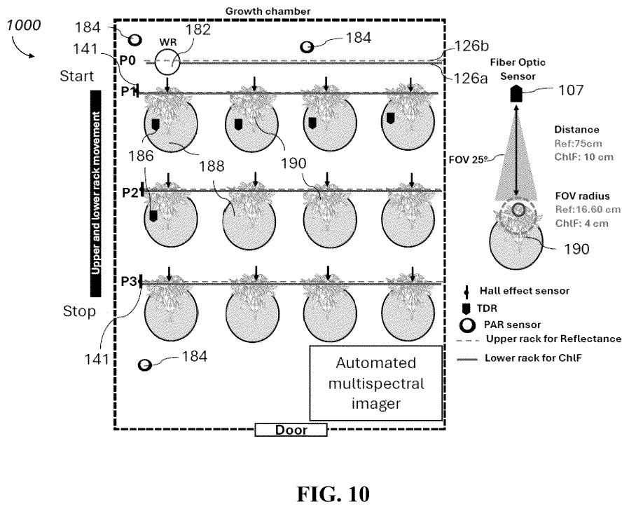

FIG. 1 illustrates a schematic diagram of a perspective view of an embodiment of a system for monitoring crops of the present patent document. Referring to FIG. 1, there is shown a perspective view of an embodiment of a crop monitoring system 100 of the present patent document. In a preferred embodiment, the crop monitoring system 100 may be an automated high-throughput phenotyping (HTP) system for monitoring crop performance. In certain embodiments, the crop monitoring system 100 may comprise a main control module 102 configured to direct operations of the crop monitoring system 100. In various embodiments, the main control module 102 may also be referred to as a controller. In some embodiments, the crop monitoring system 100 may have a light box 131, where the light module may be configured to activate a light source 133 to induce ChlF in a subject crop (see FIG. 10). In some embodiments, a light box 131 may be referred to as a light module. In a preferred embodiment, the crop monitoring system 100 may be used in plant phenotyping. The crop monitoring system 100 may be used to measure and analyze a plant's structural and functional properties, such as growth, yield, and stress adaptation, among others. In some embodiments, the crop monitoring system 100 may also be referred to as a plant phenotyping system.

In various embodiments, the crop monitoring system 100 may have a hyperspectral module 106, where the hyperspectral module 106 may be configured to collect a hyperspectral reflectance. In certain embodiments, the crop monitoring system 100 may have an imaging module 108, where the imaging module 108 may be configured to collect above-canopy RGB imagery of the subject crop. In some embodiments, the imaging module 108 may be configured to capture images in the visible light spectrum. In certain embodiments, the camera 109 may be an RGB camera that captures only three bands, broadly capturing red, green, blue wavelength. In various embodiments, a camera 109 of the imaging module 108 may be configured to capture wavelengths of light in a spectrum of about 380 to 700 nm. In other embodiments, the camera 109 may be a multispectral camera which captures a set of 3-10 discrete bands.

In various embodiments, the hyperspectral module 106 may be further configured to collect chlorophyll fluorescence emission spectra. The hyperspectral module 106 may be coupled to sensors 107 and 137. The hyperspectral module 106 may serve as the core hyperspectral sensing unit, capable of capturing reflectance over the plant canopy for comprehensive plant health data. In a preferred embodiment, the hyperspectral module 106 may be a spectrometer system such as OctoFlox, a variant of the hemispherical-conical FloX hyperspectral spectrometer system (JB Hyperspectral Devices, GmbH, Germany). In some embodiments, the hyperspectral module 106 may have the following components: a high-resolution spectrometer configured to a spectral range of about 500-900 nm with a spectral resolution of about 0.6 nm full width at half maximum (FWHM) (this configuration enables the system to obtain wide enough spectral range to simultaneously capture a variety of phenology- and stress-responsive vegetation indices that utilize NIR to visible green wavelengths, without being influenced by blue light, to facilitate nighttime chlorophyll fluorescence measurements); a multiplexer comprising a motor that aligns the spectrometer's optical inlet with one of multiple light-collecting channels, each equipped with an individual fiber optic for user configurability, or a dark measurement from a built-in dark channel; and at least one (e.g., eight in the embodiment shown in FIG. 1) 7 m long armored fiber optic cables with 200 μm core diameter may be connected to the optical multiplexer. Half of the fore-optics may be installed pointing nadir from the upper rack of the crop monitoring system 100 and used to measure hyperspectral reflectance under light conditions. The remaining channels may be installed on the lower rack to measure ChlF emission in the dark. The spectrometer may be specifically equipped with long fiber optics and ground-mounted to avoid vibration impacts on the spectrometer and greatly reduce weight that needed to be mounted on the racks or frames. In other embodiments, other spectral ranges and other spectral resolutions may be used.

The hyperspectral module 106 may use a hyperspectral point spectrometer to collect hyperspectral measurements. Hyperspectral measurements may be referred to as a technique to collect and process information across the electromagnetic spectrum to obtain the spectrum averaged over the field of view, which allows for the identification of objects and materials by analyzing their spectral signatures. While an RGB camera uses three visible light bands (red, green, and blue) to create images, hyperspectral spectrometers can examine objects and materials with much higher spectral range, ranging from 250 nm-15,000 nm, and much higher spectral resolution, from <1 nm to 20 nm per detector pixel. In various embodiments, a hyperspectral spectrometer may collect and process wavebands of 10-20 nm. The hyperspectral module 106 may use a hyperspectral point spectrometer where the hyperspectral point spectrometer averages the canopy footprint within a field of view (FOV) (as illustrated in the right side of FIG. 10). In a preferred embodiment, the hyperspectral module 106 collects 1044 individual wavebands from 500-900 nm. A hyperspectral spectrometer may collect a continuous spectrum with wavebands up to 20 nm wide and may have hundreds to thousands of bands. In a preferred embodiment, a hyperspectral spectrometer may collect a continuous spectrum with wavebands of 0.6 nm wide. In some embodiments, the hyperspectral module 106 may collect and process more than 100 discrete or individual wavebands, each waveband being <1 nm to 20 nm, of the electromagnetic spectrum. In certain embodiments, the hyperspectral module 106 may collect and process more than 1,000 discrete or individual wavebands, each waveband being <1 nm to 20 nm, of the electromagnetic spectrum.

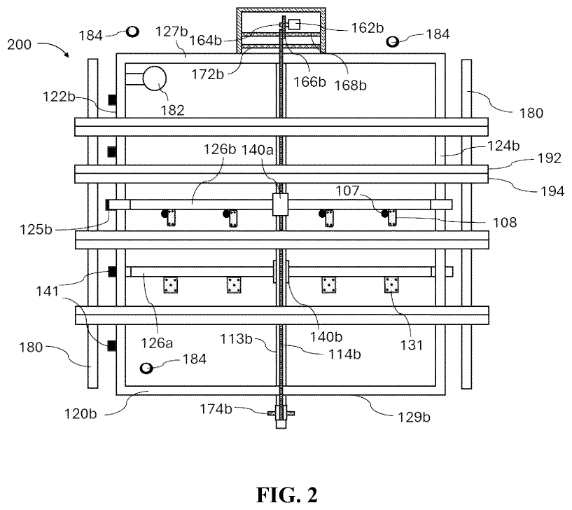

In some embodiments, the crop monitoring system 100 may have an environmental module 110, where the environmental module 110 may be configured to collect a light intensity to determine a daytime or nighttime status. In certain embodiments, the environmental module 110 may have an environmental controller to record environmental measurements. In some embodiments, the environmental module 110 may further comprise environmental sensors 184 (FIG. 2). In certain embodiments, the environmental sensors 184 may be photosynthetically active radiation (PAR) sensors, which may be used to detect a daytime or nighttime status of the crop monitoring system 100.

In some embodiments, the light box 131 may be further configured to activate a blue LED light during nighttime to induce chlorophyll fluorescence for measurements. In certain embodiments, the environmental module 110 may be further configured to collect a light intensity to determine daytime or nighttime status. In some embodiments, the environmental module 110 further comprises environmental sensors to detect at least one of a soil water availability, a temperature, and a carbon dioxide level. In various embodiments, the crop monitoring system 100 may have a movement module, where the movement module may be configured to move a first sensor rack 126a and a second sensor rack 126b carrying hyperspectral sensors, imaging modules, and light modules to appropriate positions prior to scanning. The first sensor rack 126a may be referred to as a bottom sensor rack or a lower rack, and the second sensor rack 126b may be referred to as a top sensor rack or an upper rack.

In a preferred embodiment, a crop monitoring system 100 may have a first support frame 120a, where the first support frame may comprise a first sensor rack 126a coupled to the first support frame 120a, where the first sensor rack 126a may comprise a first sensor module 131. In some embodiments, the crop monitoring system 100 may have a movement module, where the movement module comprises a first sensor rack motor 162a, wherein the first sensor rack motor 162a may be configured to move the first sensor rack 126a along the first support frame 120a from a first starting location at a front end 127a of the first support frame 120a to a first ending location at a rear end 129a of the first support frame 120a. In various embodiments, the crop monitoring system 100 may have a hyperspectral module 106, wherein the first sensor module 131 may be coupled to the hyperspectral module 106.

In some embodiments, the crop monitoring system 100 may have a second sensor rack 126b coupled to a second support frame 120b, the second sensor rack having a second sensor module 108. The movement module may further comprise a second sensor rack motor 162b, where the second sensor rack motor 162b may be configured to move the second sensor rack 126b along the second support frame 120b from a second starting location at a front end 127b of the second support frame 120b to a second ending location at a rear end 129b of the second support frame 120b. In various embodiments, the main control module 102, the hyperspectral module 106, or the environmental module 110 may be located on the first sensor rack 126a or the second sensor rack 126b.

In certain embodiments, the crop monitoring system 100 may have a main control module 102 in electrical communication with the movement module, where the controller may be configured to control the movement of the first sensor rack 126a and the second sensor rack 126b. The crop monitoring system 100 may have an environmental module 110, where the environmental module 110 may be configured to collect a light intensity from a room or surrounding environment where the system 100 is located. The main control module 102 may be in electrical communication with the environmental module 110 to determine a daytime cycle lighting condition or a nighttime cycle lighting condition based on the light intensity detected by the environmental module 110. In certain embodiments, the main control module 102 may be further configured to provide a user interface on a computer screen (not shown) and manage data.

In various embodiments, crop monitoring system 100 may be an autonomous crop monitoring system. In an embodiment where the crop monitoring system 100 is autonomous, the crop monitoring system 100 can operate on its own without direct human intervention. In certain embodiments, a user may input desired criteria into a user interface (not shown) where those instructions are given to a controller, then after receiving the set of instructions from the user, the system may operate on by itself without further human intervention.

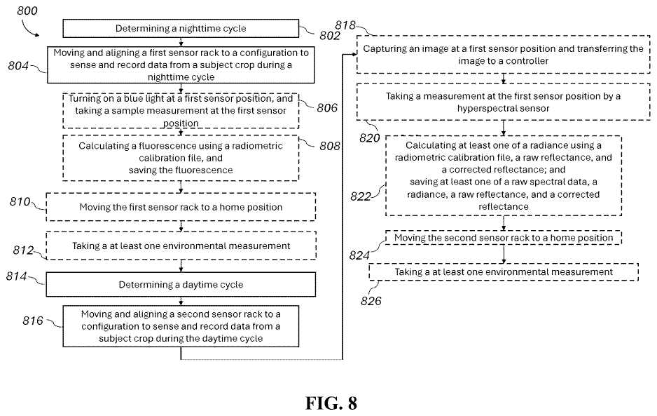

The first sensor module 131 may have a light source 133, wherein the light source 133 is configured to induce a chlorophyll fluorescence in a subject crop (see FIG. 8). The imaging module 108 may have a camera 109, wherein the camera 109 is configured to collect above-canopy red, green, and blue (RGB) imagery. In some embodiments, a camera may be referred to as a “phenocam.”

The crop monitoring system 100 may have a Hall effect (HE) sensor 141, the Hall effect sensor 141 may be coupled to the first support frame 120a. In various embodiments, the crop monitoring system 100 may have a plurality of Hall effect sensors 141. The Hall effect sensors 141 may be coupled to the support frames 120a and 120b. In some embodiments, the Hall effect sensors 141 may be coupled to the rails 122a, 122b, 124a, or 124b.

The Hall effect sensors 141 may be any Hall effect sensors known in the art. Hall effect sensors may be referred to as sensors that work by measuring the changing voltage when the sensor is placed in a magnetic field. A Hall effect sensor may be any sensor incorporating one or more Hall elements, each of which produces a voltage proportional to one axial component of the magnetic field vector using the Hall effect. When a Hall effect sensor detects a magnetic field, it is able to sense the position of a desired object. In the crop monitoring system 100, the Hall effect sensors 141 detect a magnetic field of magnets 123. When the magnet 123 is detected, the Hall effect sensor 141 sends a signal to the main control module 102 to stop the movement of the sensor rack 126a or 126b that the magnet 123 is located on.

In some embodiments, the crop monitoring system 100 may have a magnet 123 coupled to the first sensor rack 126a, wherein the Hall effect sensor 141 may be configured to sense the magnet 123 and send a signal to the main control module 102 to stop the movement of the first sensor rack 126a such that the first sensor rack 126a is over a subject crop. The first sensor rack 126a may be located below the second sensor rack 126b, such that the first sensor rack 126a is configured to travel parallel to the second sensor rack 126b between a subject crop and the second sensor rack 126b.

The crop monitoring system 100 may have a first drive shaft 164a, where the first drive shaft 164a may be operatively connected to the first sensor rack motor 162a. A first drive chain 166a may be operatively connected to the first drive shaft 164a and a first front top axle 168a. A first rack chain 114a may be operatively connected to the first front top axle 168a and a first front bottom axle 172a at a front end of the first support frame 120a. The first rack chain 114a may be operatively connected to the first sensor rack 126a. A slider 140a may be coupled to the first rack chain 114a and the first sensor rack 126a, such that the first rack chain 114a may be operatively connected to the first sensor rack 126a by the slider 140a.

The crop monitoring system 100 may have a second drive shaft 164b, where the second drive shaft 164b may be operatively connected to the second sensor rack motor 162b. A second drive chain 166b may be operatively connected to the second drive shaft 164b and a second front top axle 168b. A second rack chain 114b may be operatively connected to the second front top axle 168b and a first front bottom axle 172b at a front end of the first support frame 120a. The second rack chain 114b may be operatively connected to the second sensor rack 126b. A slider 140b may be coupled to the second rack chain 114b and the second sensor rack 126a, such that the second rack chain 114b may be operatively connected to the second sensor rack 126b by the slider 140b.

The first support frame 120a may have a first rail 122a and a second rail 124a. The second support frame 120b may have a first rail 122b and a second rail 124b. The first support frame 120a and second support frame 120b may be coupled to and supported by support legs 121 at the four corners of the support frames 120a and 120b. The system 100 may have reflective surfaces 180 running parallel to the rails 122a and 122b. The reflective surfaces 180 may be mirrors, metal, glass, or any other suitable material that reflects light. In some embodiments, the reflective surfaces 180 may be attached to walls of the growth chamber (see FIG. 10).

The first sensor rack 126a, may have at least one light box 131 coupled to the first sensor rack 126a. At least one Hall effect sensor 141 may be coupled to the first rail 122a of the first support frame 120a. At least one magnet 123 may be coupled to a first end 125a of the first sensor rack 126a, such that the magnet 123 is configured to trigger the Hall effect sensors 141 to stop the first sensor rack 126a at a location desired by a user. The first sensor rack 126a may have a light box 131 with at least one spectrometer fiber optic configured to capture a hyperspectral reflectance of a subject crop during the night.

The second sensor rack 126b, may have at least one imaging module 108 coupled to the second sensor rack 126b. A fiber optic sensor 107 may be coupled to the second sensor rack 126b. At least one Hall effect sensor 141 may be coupled to the first rail 122b of the second support frame 120b. At least one magnet 123 may be coupled to a first end 125b of the second sensor rack 126b, such that the magnet 123 is configured to trigger the HE sensors 141 to stop the second sensor rack 126b at a location desired by a user. The second sensor rack 126b may have at least one spectrometer fiber optic 107 configured to capture a hyperspectral reflectance, and at least one camera 109 configured to capture RGB images of a subject crop during the day. The fiber optic sensor 107 may be positioned at any suitable distance and location to capture a reflectance from the subject crop. In an example embodiment, the fiber optic sensor 107 is positioned at a distance from the subject crop such that the sensor can capture an image or reflectance from the subject crop in about a 23-25 degree field of view from the fiber optic sensor 107 (see FIG. 10).

The first sensor rack 126a may have a light box 131 with at least one light source 133, a light reflector 135, and a spectrometer fiber optic sensor 137 configured to capture blue light-induced chlorophyll fluorescence at night. In some embodiments, the sensor 137 may be a spectrometer fiber optic sensor. The light box 131 may have multiple light sources 133 and multiple light reflectors 135. In the embodiment shown in FIG. 1, a light box 131 has four light sources 133 with four light reflectors 135. In the embodiment shown in FIG. 1, the first sensor rack 126a has four light boxes 131. The light reflector 135 may have a concave shape, where the light source 133 is located at the center of the light reflector 135 such that the light from the light source 133 is reflected off of the reflectors 135 to further illuminate the subject crops.

A subject crop or crops that the system 100 may be monitoring may be any organism, specimen, or object that is desired to be monitored or studied. For example, the subject crop may be any biological organism such as a plant or plants. In other embodiments, the crop may be a microorganism, or organisms in a petri dish.

In the embodiment shown in FIG. 1, the second sensor rack 126b is located above the first sensor rack 126a, such that the first sensor rack 126a is configured to travel parallel to the second sensor rack 126b between a subject crop (not shown) and the second sensor rack 126b. At least one Hall effect sensor 141 may be coupled to the first rail 122b of the second frame 120b. At least one magnet 123 may be coupled to a first end 125a of the first sensor rack 126a, such that the magnet 123 is configured to trigger the HE sensors 141 to stop the first sensor rack 126a.

The light box 131 may be electrically connected to a light control module. The light box 131 may have a light source 133 and a light sensor 137. In some embodiments, the light source 133 may be a light-emitting diode (LED). In certain embodiments, the light source 133 may be a blue light. In other embodiments, the light source 133 may be a blue LED. In various embodiments, the light sensor 137 may be a fiber optic sensor. In some embodiments, the light sensor 137 may be a hyperspectral fiber optic sensor.

A hyperspectral module 106 may have a hyperspectral receiver, a multiplexer coupled to the hyperspectral receiver, and at least one hyperspectral sensor. The hyperspectral sensor may be a hyperspectral fiber optic sensor, where the hyperspectral fiber optic sensor is coupled to the hyperspectral module 106. The hyperspectral module 106 may be configured to collect a hyperspectral reflectance from a subject crop. The imaging module 108 may have a camera 109, wherein the camera 109 is electrically connected to an imaging control module. The environmental module 110 may have an environmental control module comprising of a datalogger and at least one environmental sensor, wherein the at least one environmental sensor is electrically connected to the environmental control module. In some embodiments, the environmental module may be configured to collect a light intensity. The main control module 102 is in electrical communication with the environmental module 110 to determine a daytime cycle lighting condition or a nighttime cycle lighting condition based on the light intensity from the environmental module 110. In various embodiments, the environmental module may have a photosynthetically active radiation sensor, which may be used to detect the daytime or nighttime activity of the crop monitoring system 100. In certain embodiments, the environmental module 110 may have a soil time-domain-reflectometry (TDR) probe to monitor soil moisture and temperature.

The light boxes 131 may have cables or wires 115 electrically connecting the lights boxes 131 to the main control module 102. In other embodiments, the light boxes 131 may be wirelessly connected to the main control module 102. The imaging modules 108 may have cables or wires 115 electrically connecting the imaging modules 108 to the main control module 102. The cables or wires 115 may be bundled in cable sleeves 116 so that they do not get tangled in or obstruct the racks 126a and 126b during movement. In other embodiments, the imaging modules 108 may be wirelessly connected to the main control module 102. For example, the light boxes 131 and imaging modules 108 may be connected through a wireless local area network (LAN) (e.g., Wi-Fi) to the main control module 102.

The first rack chain 114a may be coupled to a first rear top axle 174a and a first rear bottom axle at a rear end 129a of the first support frame 120a. The first rear top axle 174a may be coupled to the rack support bar 113a. The first rack chain 114a may be coupled to the first rear top gear 175a, and the first rear top gear 175a may be coupled to the first rear top axle 174a. The first rack chain 114a may be coupled to the bottom chain tensioner 176a.

The second rack chain 114b may be coupled to a second rear top axle 174b and a second rear bottom axle at a rear end 129b of the second support frame 120b. The second rear top axle 174b may be coupled to the second rack support bar 113b. The second rack chain 114b may be coupled to the second rear top gear 175b, and the second rear top gear 175b may be coupled to the second rear top axle 174b. The second rack chain 114b may be coupled to the top chain tensioner 176b.

FIG. 2 illustrates a schematic diagram of a top view of an embodiment of a system for monitoring crops of the present patent document shown in FIG. 1. In the embodiment shown in FIG. 2, a crop monitoring system 200 is shown having a top sensor rack motor 162b, the top sensor rack motor 162b operatively connected to a second drive shaft 164b, a second drive chain 166b operatively connected to the second drive shaft 164b and a second front top axle 168b, a second rack chain 114b operatively connected to the second front top axle 168b and a second front bottom axle 172b at a front end 127b of the second frame 120b, the second rack chain 114b operatively connected to the second sensor rack 126b, the second rack chain 114b operatively connected to a second rear top axle 174b at a rear end 129b of the second frame 120b. The bottom sensor rack 126a is shown below first sensor rack 126b and the second support frame 120b. In the embodiment shown in FIG. 2, the crop monitoring system 100 is shown having a white reference panel 182. The white reference panel 182 may be used to calibrate the camera 109 in the imaging module 108. In the embodiment shown in FIG. 2, the crop monitoring system 100 is shown having three environmental sensors 184. The environmental sensors 184 may be photosynthetically active radiation (PAR) sensors, which may be used to detect a daytime or nighttime status of the crop monitoring system 100. FIG. 2 shows an underside view of a growth chamber indicating the positioning of white lights sources 192 and far-red light sources 194 above the crop monitoring system. In certain embodiments, the white lights sources 192 may have a wavelength of about 400-700 nm, and the far-red light sources 194 may have a wavelength of about 700-800 nm. In some embodiments, the white lights sources 192 and far-red light sources 194 may be LEDs. The white lights sources 192 and far-red light sources 194 may be also referred to as light bars. In other embodiments, the white lights sources 192 and far-red light sources 194 may be positioned at any suitable locations to light the subject crops.

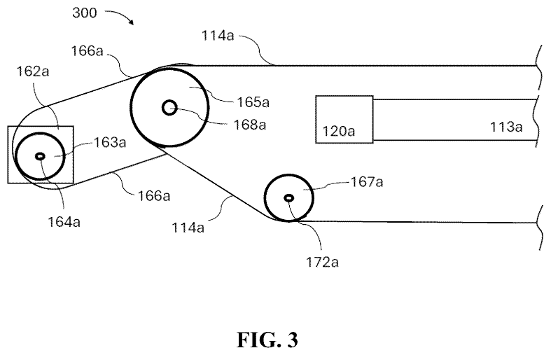

FIG. 3 illustrates a schematic diagram of a side view of a front gear system of the system for monitoring crops of the present patent document shown in FIG. 1. In FIG. 3, a front gear system 300 is shown, where the first drive shaft 164a is shown operatively connected to a first drive gear 163a. The bottom sensor rack motor 162a may be operatively connected to the first drive chain 166a by the first drive shaft 164a and the first drive gear 163a to be able to move the first drive chain 166a. A first rack chain gear 165a may be operatively connected to the first drive chain 166a and the first rack chain 114a. The bottom sensor rack motor 162a may be operatively connected to the first rack chain 114a such that the bottom sensor rack motor 162a may be capable of moving the first rack chain 114a.

The second drive shaft 164b may be operatively connected to a second drive gear 163b. The top sensor rack motor 162b may be operatively connected to the second drive chain 166b by the second drive shaft 164b and the second drive gear 163b to be able to move the second drive chain 166b. A second rack chain gear 165b may be operatively connected to the second drive chain 166b and the second rack chain 114b. The top sensor rack motor 162b may be operatively connected to the second rack chain 114b such that the top sensor rack motor 162b may be capable of moving the second rack chain 114b.

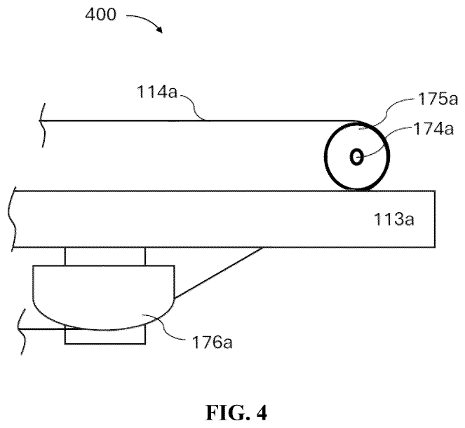

FIG. 4 illustrates a schematic diagram of a side view of a rear gear system 400 of the system for monitoring crops of the present patent document shown in FIG. 1. In the embodiment shown in FIG. 4, the first rack chain 114a is operatively connected to a first rear top axle 174a and a first rear bottom axle at a rear end 129a of the first support frame 120a. The first rear top axle 174a may be operatively connected to the rack support bar 113a. The first rack chain 114a may be operatively connected to the first rear top gear 175a, and the first rear top gear 175a may be operatively connected to the first rear top axle 174a. The first rack chain 114a may be operatively connected to the bottom chain tensioner 176a.

The second rack chain 114b may be operatively connected to a second rear top axle 174b and a second rear bottom axle at a rear end 129b of the second support frame 120b. The second rear top axle 174b may be operatively connected to the second rack support bar 113b. The second rack chain 114b may be operatively connected to the second rear top gear 175b, and the second rear top gear 175b may be operatively connected to the second rear top axle 174b. The second rack chain 114b may be operatively connected to the top chain tensioner 176b.

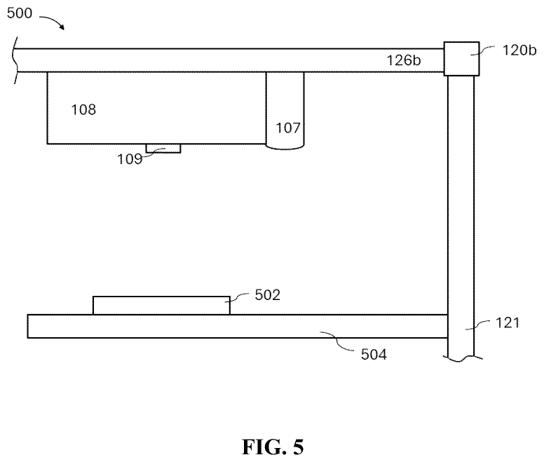

FIG. 5 illustrates a schematic diagram of a perspective view of a white reference system 500 of the system for monitoring crops of the present patent document shown in FIG. 1. In the embodiment shown in FIG. 5, a white reflectance panel 502 is disposed on a support bar 504. The white reflectance panel 502 may be located such that the imaging module 108 takes a reference measurement at the start of a daytime scan sequence. In FIG. 5, the imaging module 108 is located above the white reflectance panel 502 such that the imaging module 108 is looking at the white surface of the white reflectance panel 502 so that a reference measurement can be taken.

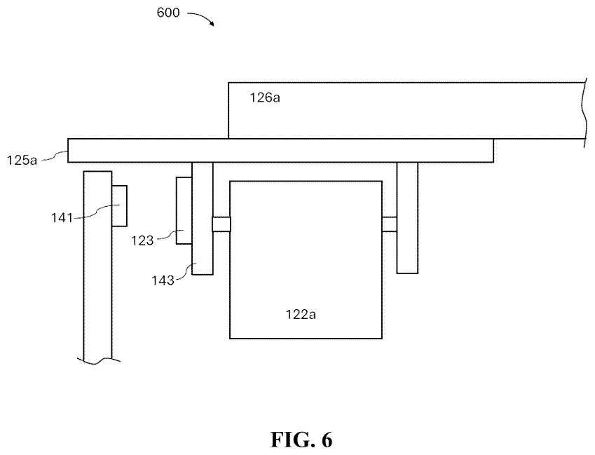

FIG. 6 illustrates a schematic diagram of a side cross sectional view of a Hall effect system 600 of the system for monitoring crops of the present patent document shown in FIG. 1. FIG. 6 shows a cross sectional view of an end of a first sensor rack 126a when the first sensor rack 126a is aligned with a Hall effect sensor 141 of the first rail 122a. The magnet 123 may be coupled to the first sensor rack 126a, such that the magnet 123 may be configured to trigger the HE sensors 141 to stop the first sensor rack 126a at a desired location. In the embodiment shown in FIG. 6, the magnet 123 is coupled to the rail guide 143. A rail guide 143 may be shown coupled to the first sensor rack 126a and the first rail 122a such that the first sensor rack 126a can slidably move along the first rail 122a.

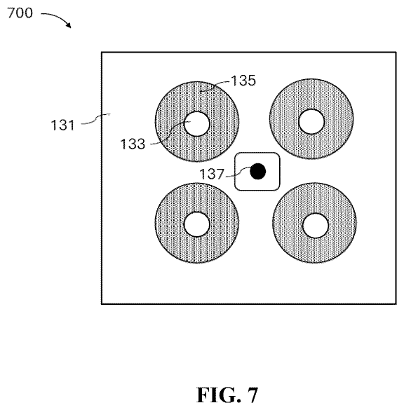

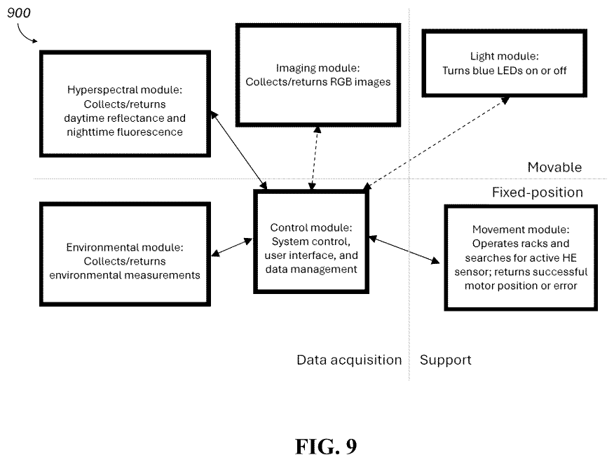

FIG. 7 illustrates a schematic diagram of a bottom view of a light system 700 of the system for monitoring crops of the present patent document shown in FIG. 1. In the embodiment shown in FIG. 7, a light box 131 is shown. The light box 131 may also be a light, a light panel, or any lighting device. The light box 131 may include LED lights. In some embodiments the light box 131 may have at least one light. In some embodiments, the light source 133 may be an LED. In certain embodiments the light source 133 may be a blue light. In a preferred embodiment the at least one light source 133 may be a blue LED light. The light source 133 may have a wavelength of 380-500 nm. In some embodiments, the light source 133 may have a wavelength of 450-495 nm. The light box 131 may have at least one light source 133. The light box 131 may have at least one reflector 135 with at least one light source 133, where the light source 133 may be located at a center of the reflector 135. The reflector 135 may have a concave shape. The light box 131 may have a sensor 137. The sensor 137 may be a fiber optic sensor capable of sensing light from the at least one light source 133 where the light is reflected off a subject plant (see FIG. 9).

FIG. 8 illustrates a process for monitoring crops of the present patent document. Referring to FIG. 8, an embodiment of a method 800 for monitoring crop performance is shown. In method 800 for monitoring crop performance, step 802 comprises determining a nighttime cycle. Step 804 comprises moving and aligning a first sensor rack to a configuration to sense and record data from a subject crop during a nighttime cycle. Step 806 comprises turning on a blue light at a first sensor position, and taking a sample measurement at the first sensor position. Step 808 comprises calculating a fluorescence using a radiometric calibration file, and saving the fluorescence. Step 810 comprises moving the first sensor rack to a home position. Step 812 comprises taking a at least one environmental measurement. Step 814 comprises determining a daytime cycle. Step 816 comprises moving and aligning a second sensor rack to a configuration to sense and record data from a subject crop during the daytime cycle. Step 818 comprises capturing an image at a first sensor position and transferring the image to a controller. Step 820 comprises taking a measurement at the first sensor position by a hyperspectral sensor. Step 822 comprises calculating at least one of a radiance using a radiometric calibration file, a raw reflectance, and a corrected reflectance and saving at least one of a raw spectral data, a radiance, a raw reflectance, and a corrected reflectance. Step 824 comprises moving the second sensor rack to a home position. Step 826 comprises taking a at least one environmental measurement. In FIG. 8, although the steps of method 800 are listed in a particular order, in other embodiments, the steps of method 800 may occur in other orders as well.

The following are software operation routines used in an example embodiment of the crop monitoring system of the present disclosure. Although the steps herein are listed in a particular order, in other embodiments, the steps may occur in other orders as well.

Normal Operation Routine. This is the operation routine of controller_main.py which essentially operates the crop monitoring system.
1. Initialize communication with all sub-modules (hyperspectral, imaging, motor, datalogger, light). Retry up to 5 times with 5-second wait if needed for delayed booting (e.g. on phenocams), if all retries fail, abort script.
2. Load user-defined schedule from config.ini
3. Load user-defined calibration file from config.ini
4. Load user-defined correction factor file from config.ini
5. Check user-defined correction factor file structure is valid, otherwise abort script.
6. Generate new empty files for the day, labeled by date.
7. If time to take measurement, check light condition:
   a. If daytime:
      i. Take white reference panel radiance measurement at home position. Normalize by integration time.
      ii. For rack positions 1-n (user-defined):
         1. Move upper rack to first target position.
         2. For sensor positions 1-4:
         a. Take photo at sensor position. Transfer image file from phenocam to main controller.
         b. Take sample measurement at target position.
         c. Calculate radiance using radiometric calibration file.
         d. Calculate raw reflectance (DNSam/ITSam)/(DNWR/ITWR).
         e. Calculated corrected reflectance (reflectance*correction_factor from file).
         f. Save raw spectral data, radiance, raw_reflectance, corrected_reflectance.
      iii. At end of all rack measurements, move rack to home position.
      iv. Take environmental measurements for the whole room from the datalogger and save to file.
   b. If nighttime:
      i. For rack positions 1-n (user-defined):
         1. Move lower rack to first target position.
         2. For sensor positions 1-4:
         a. Turn on blue LEDs at sensor position.
         b. Take photo at sensor position. Transfer image file from phenocam to main controller.
         c. Take sample measurement at target position.
         d. Calculate fluorescence (radiance) using radiometric calibration file.
         e. Save raw spectral data, fluorescence.
      ii. At end of all rack measurements, move rack to home position.
      iii. Take environmental measurements for the whole room from the datalogger and save to file.
8. Countdown to next scheduled measurement.
9. Repeat steps 7-8 as scheduled.

Correction Factor Generation Routine. A correction factor file must be generated by the user before normal operation routine can be performed for correct reflectance values. Generate this file by running cf_generator.py.

The sequence of operations performed by cf_generator.py are as follows:
1. Initiate communication with hyperspectral and motor modules. Move upper rack to home position if not already home.
2. Request user input to define:
   a) number of scans to average (“nscans”; value can be 1-n, realistically 10 max is likely more than sufficient; this is to average out instrument/chamber noise), and
   b) number of Hall Effect positions to calibrate (“nHEs”; value can be 1-8; maximum is defined by number of Hall effect sensors installed in the motor module).
3. Acknowledges user inputs and displays instructions on screen which motor and fiber position numbers will be activated and in which direction to orient the user. This is important since the user will need to manually place the white reference panel (mounted on a tripod) beneath each position to be calibrated.
4. Ask the user if they are ready to scan at the home position and confirm by pressing Enter. This user input step allows the user to position the reference panel appropriately at their own speed as needed.
5. Scan white reference at the home position nscans times, normalize by integration time and store average value. (DNWR_home/ITWR_home)
6. For rack positions 1-nHEs:
   a. Move upper rack to target position.
   b. For sensor positions 1-4:
      i. Ask the user if they are ready to scan at position (rack, sensor) and confirm by pressing Enter. This informs the user where to place the white reference panel next and gives them time to move it.
      ii. Scan white reference at (rack, sensor) position nscans times, normalize by integration time and store average value. (DNWR_Sam/ITWR_Sam).
      iii. Calculate correction factor at this position as the ratio of the home WR/target WR.

$$correction\_factor = ( DNWR\_home / ITWR\_home ) / ( DNWR\_Sam / ITWR\_Sam ) .$$

The final corrected reflectance calculated in the main operation routine will therefore be:

$$corr\_refl = ( DNSam / ITSam ) / ( DNWR\_home / ITWR\_home ) * ( DNWR\_home / ITWR\_home ) / ( DNWR\_Sam / ITWR\_Sam ) = ( DNSam / ITSam ) / ( DNWR\_Sam / ITWR\_Sam ) .$$

iv. Collect the correction factors labeled by motor and sensor position in a list (array size [nHEs*4, 1026]; columns are labeled as motor_position, sensor_position, and detector pixels 1-1024).
7. Convert the correction factor list into a Pandas DataFrame and save it to a comma separated value file, labeled with the date and time in case multiple files are generated on the same day. A set of files could be generated under different light conditions if multiple light levels or different light qualities were used in the same experiment, and the appropriate file could be applied under a given detected light condition.
8. Remind user to update config.ini to use the newly generated config file.

### User Configuration Options.

1. Schedule of measurements. Define in config.ini in the [SCHEDULE] section. Options include explicitly specifying time of day and how many days of the week to measure, or how many days of the week to measure and a recurring interval in minutes.
2. Modules to activate (either hyperspectral, RGB imaging, both, or neither). Define in config.ini in the [GENERAL] section.
3. Location of Hall Effect sensors (where the sensor racks will stop). Position manually.
4. Number of Hall Effect sensors (how many positions will be in the chamber). Define in config.ini in the [GENERAL] section. There need to be equal numbers on lower and upper rack for current functionality, so only one integer input is supported.
5. Duration of blue light application before chlorophyll fluorescence measurement. Define in config.ini in the [GENERAL] section. This is mainly for experimentalists to test different blue light application effects.
6. Calibration files. Define in config.ini in the [CALIBRATION] section. These should be updated by the end user routinely to ensure the hyperspectral spectrometer is calibrated (every 1-2 years or any time there is service on the instrument) and that the chamber correction factors are correct (before the beginning of an experiment or any time there is service on the growth chamber/light banks or change in position of the racks/targets during an experiment). The radiometric calibration file can be user generated through the hyperspectral spectrometer GUI. The correction factor calibration file is user generated by running cf_generator.py and following instructions (see above section).
7. Type and number of datalogger sensors. This is user configurable and needs extra configuration between the datalogger software and the main controller, but once they are done the communication and data acquisition will be triggered by main controller. The crop monitoring system software does not include easy modification of the datalogger and this is not planned to be implemented at present because half of the code that needs to be edited is datalogger code, which may differ based on user. The crop monitoring system lays the groundwork to communicate with a datalogger on a scheduled basis, thus supporting flexible environmental data collection for end users.

The crop monitoring system's data management approach are critical components to consider for high throughput phenotyping systems because they generate so much data over time (images, log files, data files). These are the supported functions:

Enable directly saving to a physical external storage drive instead of on the main controller's SD card, for expandable (and removable) storage space.

Remote backups to network location and periodic removal of files from main controller to maintain consistent free storage space.

Reference will now be made to an experiment, including the system used and the results of the experiment. The embodiments, systems, apparatus, and methods disclosed herein are non-limiting examples only.

The crop monitoring system disclosed herein was developed to address the challenges by integrating cutting-edge technologies for continuous and comprehensive crop monitoring in controlled environments, with the unique capability to perform both day and night measurements over multiple plant canopies. The crop monitoring system of the present disclosure may also be referred to as the “PhenoGazer.” Compared to existing high-throughput phenotyping systems, the crop monitoring system of the present disclosure offers several advantages. Its portability and flexibility make it suitable for use in diverse experimental settings, including walk-in growth chambers and smaller-scale facilities. The integration of blue LED-induced chlorophyll fluorescence measurement is a unique feature that allows for detailed nighttime analysis of chlorophyll fluorescence. Furthermore, the use of cameras (e.g., a Raspberry Pi camera) and Python-based automation provides a cost-effective solution for high-resolution imaging and data management, enhancing the system's accessibility and usability. To validate the efficacy of the crop monitoring system, an experiment was conducted on soybean plants subjected to different water and temperature treatments. These experiments were performed in a walk-in controlled-environment chamber designed to simulate various environmental conditions. The objective of building the crop monitoring system is to develop a versatile, cost-effective, and portable phenotyping system that can be deployed in various experimental settings, offering detailed insights into plant health and development with minimal human intervention. This experiment aimed to detail the design and functionality of the crop monitoring system, present an example of experimental results, and discuss the application of the crop monitoring system to enhance the understanding of crop responses to environmental conditions and supporting optimized crop performance.

High throughput phenotyping for crop monitoring at both leaf and canopy scales is essential for understanding plant responses to various stresses. The crop monitoring system, a high-throughput phenotyping system, enhances crop monitoring in controlled environments by integrating a portable hyperspectral spectrometer with eight fiber optics, four Raspberry Pi cameras, and blue LED lights. This system allows for comprehensive assessment of plant health and development. The crop monitoring system features automated moveable upper and lower racks for continuous measurements. The lower rack, equipped with four blue LED lights and spectrometer fiber optics, captures blue light-induced chlorophyll fluorescence at night. The upper rack, carrying four spectrometer fiber optics and cameras, captures hyperspectral reflectance and RGB images during the day. This dual capability enables detailed evaluation of plant phenology, stress responses, and growth dynamics throughout the entire crop growth cycle. Fully automated and managed by a Raspberry Pi running Python scripts, the crop monitoring system ensures precise control and data acquisition with minimal human intervention. Additionally, the system includes continuous measurements through a datalogger to acquire photosynthetically active radiation (PAR), soil moisture and temperature, and features expansion capability for additional analog or digital sensors as desired by end users. To test the system, soybean plants were grown under varying water treatments as well as diseased plant to monitor growth and stress responses. The crop monitoring system successfully phenotyped plants under different conditions in a walk-in growth chamber. By combining chlorophyll fluorescence, hyperspectral reflectance based five vegetation indices, the crop monitoring system of the present disclosure represents a significant advancement in plant phenotyping technology, enhancing our understanding of crop responses to environmental conditions and supporting optimized crop performance in research and agricultural applications.

In one example embodiment, the crop monitoring system comprises a main control module, which provides the user interface and directs system operations and data management; a hyperspectral module, which collects hyperspectral reflectance and chlorophyll fluorescence emission spectra; an imaging module, which collects above-canopy RGB imagery, which is used for downstream phenology analysis and position accuracy analysis; an environmental module, which collects light intensity to determine day or night status along with any other user-determined environmental sensors such as soil water availability, temperature, CO2 levels, among others; a light control module, which activates blue light during nighttime to induce chlorophyll fluorescence for measurements; and a movement module, which moves sensor racks carrying the hyperspectral, imaging and light modules to appropriate positions prior to scanning.

### Materials and Methods

Hardware design. Compared to previous high-throughput phenotyping (HTP) systems, which were primarily designed for field and greenhouse conditions, the crop monitoring system is specifically designed for growth chamber conditions, particularly walk-in chambers. This design allows for the phenotyping of multiple plant canopies under both day and night conditions while providing the experimenter full control of all abiotic parameters such as light, temperature, CO2, humidity, water and nutrient availability. In an example embodiment, the crop monitoring system is constructed with a rectangular horizontal footprint measuring 3.05 m long and 1.82 m wide (see FIG. 1). The system comprises two identical aluminum rail frames connected to form the upper and lower racks, linked together by 0.76 m aluminum rail sections. The frame structure is supported by four adjustable legs, allowing the height to be modified before or during experiments for different canopy heights. The frame can also be extended or shortened to accommodate different floor setups based on experimental requirements. On each frame, a central rail supports a sensor rack carrying sensors on either side. The sensor rack is attached to the central rail using a slider mechanism that allows it to move forward and backward over the plant canopies. The upper and lower racks are equipped with four fiber optics for hyperspectral reflectance and chlorophyll fluorescence acquisition. The upper rack is additionally equipped with four Raspberry Pi cameras for canopy imaging, and the lower rack is equipped four blue LED light boxes for nighttime measurements. All components of the crop monitoring system are controlled via a main control module. Systems operations are fully automated using Python, eliminating the need for human intervention aside from initial experimental configuration and system calibrations. Two stepper motors (e.g., HT23-598, Applied Motion Products, Morgan Hill, CA, USA), placed at the head of the upper and lower frames, drive the sensor racks forward and backward using a combination of active gears, idle gears, and chains with a chain tensioner to maintain proper tension. Stepper motor drivers (e.g., STR2, Applied Motion Products) are used to control the motors independently when instructed from the main control module.

To control the sensor bar movements and ensure precise stopping over specific plant canopy positions, industrial Hall effect (HE) sensors are installed at user-selected positions along the rails. Magnets connected at one end of each sensor rack trigger the HE sensors to stop the sensor rack with high accuracy. These sensors are also connected to the Raspberry Pi computer and operate according to pre-determined schedules defined by the user in a configuration file. This innovative design of the crop monitoring system, tailored specifically for controlled growth chamber environments, allows for fully user-customizable, comprehensive day and night monitoring of plant canopies, enhancing the capability to phenotype crops under various conditions and stresses.

FIG. 9 illustrates a schematic diagram of some modules of the system for monitoring crops of the present patent document. Core functional modules are shown integrated into the crop monitoring system 900. Modules are displayed to indicate movable (top) and fixed (bottom) positioning, and data acquisition (left) and support (right) functions. Dashed arrows indicate wireless communication. Solid arrows indicate wired serial communication.

### Sensors and Communications.

The crop monitoring system incorporates a combination of sensors to facilitate detailed plant phenotyping. Core components of the crop monitoring system can be categorized into six modules, encompassing the main system control module, three data acquisition support modules (imaging, hyperspectral, and environmental measurements) and two functional support modules (light and movement control) (see FIG. 9). All the crop monitoring system modules are programmed in Python, with supporting software in C for the movement module and CRBasic for the environmental module. In other embodiments, other computer programming languages may be used.

### Main Control Module: System Control and User Interface.

A microcomputer (e.g., Raspberry Pi 3B+, Raspberry Pi Ltd., Cambridge, UK) serves as the core of the main control module, which may be referred to as the “HTPController”. The microcomputer features four USB ports, one Ethernet port, and built-in Wi-Fi chip. The HTPController is accessible by users through secure shell protocol (SSH) to interact with a command-line interface or virtual network computing (VNC) to interact with a graphical user interface. The latter is preferable for shared operation among multiple users. On power up, the HTPController broadcasts a local Wi-Fi hotspot, providing local access for system monitoring and data transfer. If the HTPController is connected by Ethernet to the user's network, the crop monitoring system will also be remotely accessible.

The hyperspectral, environmental and movement modules are connected directly to the HTPController using USB serial cables to transmit and receive data according the baud rates required by each supporting module (57600 for the hyperspectral module; 115200 for the environment and movement modules). Because the light and imaging modules are rack-mounted, these modules communicate wirelessly with HTPController through the Wi-Fi hotspot.

### Hyperspectral Module: Acquisition of Reflectance and Fluorescence Spectra.

The Hyperspectral module uses a hyperspectral spectrometer system (e.g., OctoFlox). The hyperspectral spectrometer system serves as the core hyperspectral sensing unit, capable of capturing reflectance over the plant canopy for comprehensive plant health data. OctoFlox is a variant of the hemispherical-conical FloX hyperspectral spectrometer system (JB Hyperspectral Devices, GmbH, Germany). The spectrometer may comprise the following main components: 1) A high-resolution spectrometer (e.g., QE Pro, Ocean Optics, Orlando, FL, USA) configured to a spectral range of 500-900 nm with a spectral resolution of 0.6 nm FWHM. This configuration enables the system to obtain wide enough spectral range to simultaneously capture a variety of phenology- and stress-responsive vegetation indices that utilize NIR to visible green wavelengths, without being influenced by blue light, to facilitate nighttime chlorophyll fluorescence measurements. 2) The spectrometer is connected to a multiplexer comprising a piezoelectric motor that aligns the spectrometer's optical inlet with one of eight light-collecting channels, each equipped with an individual fiber optic for user configurability, or a dark measurement from a built-in dark channel. 3) Eight identical 7 m long armored fiber optic cables with 200 μm core diameter are connected to the optical multiplexer. Four of the fore-optics are installed pointing nadir from the upper rack of the crop monitoring system and used to measure hyperspectral reflectance under light conditions. The remaining four channels are installed on the lower rack to measure chlorophyll fluorescence emission in the dark. The spectrometer was specifically equipped with long fiber optics and ground-mounted to avoid vibration impacts on the detector and greatly reduce weight that needed to be mounted on the rack.

When powered on, the spectrometer system is initialized in manual mode. Hyperspectral measurements are triggered by the main control module sending the request measurement command (e.g., “mc 1” to measure channel 1) to the spectrometer over a wired USB serial communication. Nighttime measurements, defined by the datalogger PAR sensor returning <50 μmol m-2 s-1, are obtained from channels 1-4, which are mounted on the lower rack. Daytime measurements are obtained from channels 5-8. Depending on light status, data responses are automatically sorted and labeled with motor position. New files are created each day and labeled by date. Each daytime scan sequence begins with a reference measurement from channel 5, collected over a mounted white Spectralon calibrated reflectance panel with 99% reflectance. Daytime reflectance measurements are calculated as the ratio between sample measurements and the reference measurement at each pixel, each normalized by the integration time of the respective measurement, as follows: (DNS(p)/ITS)/(DNR(p)/ITR), where DNS and DNR represent raw digital numbers returned from the spectrometer for sample or reference, respectively; p represents pixel number; and ITS and ITR represent integration times obtained at sample or reference positions, respectively. Nighttime fluorescence measurements are calculated in radiance units after correcting for detector nonlinearity and dark-current noise and applying a pixel-specific radiometric calibration (as described in Gu et al., 2019). Reflectance, fluorescence, raw data and dark-current scans returned from the spectrometer are saved as separate files.

### Imaging Module: Acquisition of RGB Images.

Four phenology-monitoring cameras, which may be referred to herein as “phenocams,” are installed on the crop monitoring system along the upper rack. Each phenocam is constructed from lightweight, low-cost Raspberry Pi Zero 2 W units, each equipped with a Raspberry Pi Camera v3. The Pi Camera is capable of high-resolution (11.9 megapixel, 4608×2592 pixel sensor resolution) RGB imaging and is integrated into the system to analyze plant morphology as well to monitor the accurate positioning of the sensor over the plant canopy. Version 3 of the Pi Camera features a motorized auto-focusing lens as well as an advanced image sensor, which is designed to capture high-quality images with improved color fidelity and noise reduction in low light conditions. The Pi Camera natively features 66° horizontal and 41° vertical fields of view (FOV), providing sufficient coverage to observe the target area in experimental setups.

On startup, the main control module broadcasts a local Wi-Fi hotspot. The four phenocams, each assigned unique static IP addresses, are configured to communicate headlessly with the Wi-Fi hotspot. When the crop monitoring system automation is initiated, all four phenocams are pinged to ensure cameras are active. During daytime and nighttime hyperspectral scans, image acquisition is requested from the phenocam corresponding to the currently scanning position using SSH. Images are then immediately transferred to the main control module through a secure copy protocol (SCP). Image files are labeled with the date, time, and camera identifier.

### Environmental Module: Acquisition of Light and User-Defined Environmental Sensors.

Environmental conditions are continuously monitored during operation using a datalogger (e.g., CR1000, Campbell Scientific, Inc., Logan, UT, USA) connected to the main controller by a serial communication cable. The datalogger is natively equipped with a photosynthetically active radiation (PAR) sensor, which is used to detect the daytime or nighttime activity of the system. This autodetection enables the experimentalist to set custom light and sampling routines and automate action of the reflectance and fluorescence racks accordingly. The datalogger can be additionally equipped by the user with a range of sensors to measure temperature, humidity, light intensity, pressure, etc. The datalogger natively supports up to 8 analog devices, which can be expanded with a multiplexer, as well as up to 36 (A-Z, 0-9) uniquely addressed SDI-12 digital sensors. For the purposes of this study, a datalogger was equipped with one PAR sensor and five soil time-domain-reflectometry (TDR) probes (e.g., TDR-315H, Acclima Inc., Meridian, ID, USA) to monitor soil moisture and temperature and demonstrate system function.

When the crop monitoring system automation is initiated, the main control module sends a communication request to the datalogger and awaits an acknowledgement response to ensure datalogger communications are active. The datalogger communicates with the main control module using a Modbus protocol. Each desired environmental sensor measurement is assigned to a Modbus register. The main control module can request a single register position (for example, to acquire PAR to determine daytime status), or the full Modbus register (for example, to record all environmental sensors at once).

### Light Module: Operation of Blue LEDs.

Custom-designed blue light LED units were strategically deployed on lower rack to provide controlled light during nighttime chlorophyll fluorescence measurements. Blue LED lights are primarily used to provide a specific wavelength of light that is important for studying plant physiological processes. Blue light, typically around 450-495 nm in the visible light spectrum, is essential for regulating processes such as phototropism, stomatal opening, and chlorophyll absorption. This makes blue LEDs particularly useful for experiments focusing on these aspects of plant biology. In the system, as indicated by the diagram, the blue light module is strategically positioned to ensure uniform illumination over the targeted plant canopies being studied, with an aperture at the center of the LED enclosure through which the spectrometer fiber optic obtains measurements of the illuminated target below.

A Raspberry Pi Zero 2 W acts as the light module controller. This unit is assigned a static IP address and configured to communicate headlessly with the Wi-Fi hotspot broadcast by the main control module. The light module controller is directly connected by GPIO pins to a set of four relay switches that activate when requested, supplying power to the blue light LED boxes mounted on the lower rack. The activation or deactivation of LEDs is performed by the light module controller via a Python script when requested via SSH from the main control module. When the crop monitoring system automation is initiated, the light module controller is issued a command to switch off any lights if on, in case prior system operations were interrupted in a lights-activated state.

### Movement Module: Sensor Rack Positioning.

Hall effect (HE) sensors are utilized for the precise positioning of the upper lower racks over the plant canopies, ensuring consistent and accurate measurements across all experimental units. Hall Effect sensors are a type of transducer that vary their output voltage in response to changes in magnetic field. These sensors utilize a thin strip of metal with a current applied along it, combined with a magnetic field perpendicular to the current. When the magnetic field is present, the electrons in the current are deflected toward one side, creating a voltage difference across the opposite sides of the strip, known as the Hall voltage. This phenomenon is the basis for how Hall Effect sensors detect and measure magnetic fields [24]. These sensors can be designed to produce a digital output (on/off), where the sensor switches when the magnetic field exceeds a certain threshold, or an analog output that provides a continuous voltage level based on the strength of the magnetic field. In the context of High Throughput Phenotyping (HTP) systems, Hall Effect sensors can be used for precise positioning and control of automated movement. This precision is crucial for consistent data acquisition and repeatability of measurements over the duration of experiments.

In the crop monitoring system, positional commands from the main control module are interpreted by the movement module. The core of the movement module comprises two synchronized units, HTPController and HTPLED, which communicate through socket commands initiated on program startup. When movement of sensors on a given rack is requested during a measurement routine, HTPController sends a “move <rack> <direction>” command to HTPLED, which in turn defines the direction by supply a high or low pin to the stepper driver, and activates motor movement for the designated rack by supplying high and low step signals to the corresponding motor stepper driver.

The stopping position for the motor is handled by HTPController, which receives signals from the HE sensors (e.g., 54100-17X-02-A, Littelfuse Inc., Chicago, IL, USA) through GPIO pins. Eight pins are assigned to HE sensor positions along the lower fluorescence frame, while another eight pins are assigned to HE sensor positions along the upper reflectance rack. When activated by proximity to a magnet, the data signal from a HE sensor at a given position shift from high to low voltage. HTPController checks for whether the active HE sensor is at the intended target position (stop requested with a success message), activated en route to the target position (ignored), or activated after the target position (stop requested with an error message). Stop requests are submitted by HTPController as “interrupt <rack>” to HTPLED. In response, HTPLED activates an interrupt pin on the stepper driver, forcing the motor off and setting the motor into an idle state until the next move command is requested.

Motor movement routines are determined based on daytime status. During daytime, the upper rack is moved to target positions to scan reflectance and capture overhead canopy images within the chamber. During nighttime, the lower rack is moved to target positions to scan fluorescence, while the upper rack also moves to capture overhead canopy images and ensure rack position accuracy within the chamber. When the crop monitoring system automation is initiated, the movement module controller is issued a command to return both upper and lower racks to home position, in case prior system operations were interrupted while the sensor rack was positioned away from home.

### User-Specified Configurations.

The user can submit instructions to the crop monitoring system via a simple user configuration file. Within this file, the user can define measurement schedule, either by time of day in HH:MM format (e.g., 08:00, 11:30, 12:30, 13:30, 15:30), or by defining the frequency in minutes and the number of days to repeat weekly (e.g., 15 minute intervals for a full day every 2 days). The measurement time indicates the time at which a full room scan will be performed. The user can also select whether to toggle off the hyperspectral and/or imaging modules as desired. The environmental module cannot be disabled through the configuration file since it is needed to detect daytime status, although the user can modify and expand the number and type of environmental sensors connected to this module. The user also defines the number of motor positions designated by the user within the crop monitoring system's frame (up to 8 currently supported, for a total of 32 scannable positions). These positions are selected by the user by sliding the HE sensors along the frame of the crop monitoring system such that they will stop the rack above the desired target plant or canopy position during scanning. The user can also define the duration of the blue light prior to nighttime fluorescence scanning, for experimental purposes. Lastly, the user can direct the crop monitoring system to the latest radiometric calibration file of the hyperspectral spectrometer, such that fluorescence spectra can correctly be converted to radiance units.

### Scanning Routine Operations.

When the crop monitoring system automation is initiated, connections are initialized with all support modules. During the initialization step, the main control module determines if any support modules are returning communication errors, allowing for up to five re-connection attempts in case any of the modules are still booting up when contact is initiated (for example following a power interruption). A sequence of scans, based on the number of HE positions and time points defined by the user in the configuration file, are then automatically acquired by the system. The daily scanning routine for reflectance (R) and fluorescence (F) measurements is systematically designed to capture detailed physiological data from plants. The sequence of steps each day involves positioning the sensors accurately to measure both R and F at predetermined spots within the experimental setup. Below, the experiment will refer to the complete sequence of scans on a given rack as a full-room scan (having 4 channels×n user-defined HE positions).

Prior to each full-room scan, light intensity is requested from the environmental module to determine daytime status.

Detection of sufficient light within the chamber automatically activates the daytime reflectance measurement routine. This involves an initial measurement of a white reflectance panel by channel 5 at the upper rack home position at the back of the chamber, which serves as the reference for that series of full-room measurements. The movement module then positions the upper rack at the first rack position, near the back of the chamber. The hyperspectral and imaging modules then acquire measurements sequentially from left to right along the sensor rack, recording sample spectra and images from corresponding channels and phenocams. After each sample spectrum acquisition, reflectance is calculated and all spectra and images are stored their respective data folders. After all four positions have been acquired along the sensor rack, the movement module positions the upper rack at the next designated rack position moving toward the front of the chamber, until the user-defined maximum rack position is attained. After the full-room scan is completed, the upper rack is moved back to the home position, and the environmental module returns all environmental measurements collected from the room.

Detection of darkness automatically activates the nighttime chlorophyll fluorescence measurement routine. The movement module positions the lower rack at the first rack position near the back of the chamber. The upper rack is then moved to the first rack position directly over the lower rack. Subsequently, the lower rack activates the light module at the first scanning position along the sensor rack for a user-specified duration. During the light activation period, the imaging module is triggered on the upper rack to monitor movement position accuracy. At the end of the blue light activation period, the hyperspectral module collects a chlorophyll fluorescence measurement which is converted to radiance units and saved. The light at that sensor position is then switched off and the next sensor position is activated. After fluorescence spectra at all four positions along the rack have been acquired, both racks then proceed to the next rack position and the sequence repeats until the user-defined maximum rack position is attained. After the full-room scan is completed, both racks move back to the home position, and the environmental module returns all environmental measurements collected from the room.

Between scheduled full-room scans, the system waits in an idle state with all lights off and both racks in home position, and a countdown of seconds to the next scheduled scan is presented. Should power issues interrupt operations, the main control module automatically restarts the crop monitoring system program using a bash script unless instructed otherwise.

### Evaluation of Consistency and Precision.

To ensure the consistency and precision of the crop monitoring system, the experiment verified the positional accuracy of the sensor rack. This analysis addresses the accuracy of the system's movement and alignment capabilities, crucial for ensuring that the phenotyping data are collected from the correct locations. Multiple plant targets were strategically placed on the ground within the scanning area, and the system was programmed to repeatedly measure these targets over the course of three days. This approach allowed for the assessment of both short-term repeatability and long-term reliability of the sensor outputs. Fourteen reflectance measurements across each day were recorded over twelve chamber positions (comprised of four fiber channel positions and three rack positions) over the course of three days. The experiment utilized the images collected during a typical crop monitoring system reflectance measurement routine, collecting a total of 504 images. The experiment then evaluated positional accuracy within the images to monitor and document any drift or deviation in HE sensor performance.

The positional accuracy estimation was conducted as follows: The first image collected each day at a given channel and rack position was identified as the reference image, with the center of the image identified by convolving the image against itself; the center presents as the brightest pixel in the image. All subsequent sample images collected on the same day for each channel and rack position combination were then convolved against its respective reference image, and the distance between the position of the brightest spot in the new convolved image and the reference image center was calculated along the x-axis (parallel to the sensor rack) as well as the y-axis (perpendicular to the sensor rack, i.e. along the direction of motor movement). Lastly, the shifted distance in pixels was converted to distance in centimeters (30 px=1 cm) based on an image from the system containing a ruler placed on the ground in the field of view, to quantify the actual positional error of the camera (representing a fixed sensor on the rack) relative to a ground target. To optimize image processing performance, convolution was performed using fast Fourier transform (FFT) in Python via the fftconvolve function in SciPy (Virtanen et al. 2020). The collected data from these repeated measurements were also statistically analyzed to identify any systematic errors or random variations, and precision was quantified by standard deviation and coefficient of variation (the ratio of the standard deviation to the mean). Inaccurate positioning may thus be determined from the positional alignment by accuracy or precision metrics, with measurements acquired during bad positions flagged for removal.

FIG. 10 illustrates another schematic diagram of the system for monitoring crops of the present patent document. FIG. 10 illustrates a schematic of a scanning layout of plants and sensors of a crop monitoring system 1000 from a top-down view of a growth chamber. Solid lines and dashed lines indicate positions of moving bars of lower racks 126a and upper racks 126b for ChlF and reflectance measurements over the plant canopies of the subject crop 190 from position P1 to P3 respectively. Arrows indicate the positions of sensors 107, 137. In the embodiment shown in FIG. 10, five soil time-domain-reflectometry (TDR) probes 186 are shown to monitor soil moisture and temperature and demonstrate system function. The subject crops 190 may be located in soil 188. The soil time-domain-reflectometry probes 186 may be located in soil 188. A fiber optic sensor may be positioned at any suitable distance and location to capture a reflectance from the subject crop 190. In an example embodiment, the fiber optic sensor 107 is positioned at a distance from the subject crop 190 such that the sensor 107 can capture a reflectance from the subject crop 190 in about a 25 degree field of view (FOV) from the fiber optic sensor 107 (see FIG. 10). The hyperspectral module 106 may use a hyperspectral point spectrometer where the hyperspectral point spectrometer averages the canopy footprint within a field of view (as illustrated in the right side of FIG. 10). In some embodiments, the first sensor rack 126a may be positioned about 10-25 cm above the canopy of the subject crop. In a preferred embodiment, the light box 131 is positioned about 10 cm above the canopy of the subject crop. In certain embodiments, the second sensor rack 126b may be positioned about 75 cm above the canopy of the subject crop. In other embodiments, other distances of the first sensor rack 126a or the second sensor rack 126b from the canopy of the subject crop may be used. In other embodiments, the distance of both the first sensor rack 126a and the second sensor rack 126b can be adjusted as needed to change the FOV/spot size for measurements from any sensors or cameras.

### System Validation Experiment.

The crop monitoring system was installed in a walk-in growth chamber. A controlled-environment study was conducted within this chamber to demonstrate the functionality of the crop monitoring system, as illustrated in the provided schematic (FIG. 10). This schematic includes top-down view of the scanning layout, detailing the positions where reflectance and fluorescence are measured relative to the locations of the PAR and soil moisture sensors. The top-down view in the schematic shows the layout of plant pots and sensor tracks, indicating specific spots where R/F measurements were taken. It also shows the locations of environmental sensors, such as soil moisture sensors to monitor water content at different points within the setup. These measurements are taken at consistent times throughout the day to track diurnal variations in plant responses. This structured approach ensures that all environmental variables and plant responses are thoroughly documented, providing a robust dataset for analyzing the impact of different treatments on plant health and development.

Soybean plants were used to evaluate the robustness of a High Throughput Phenotyping (HTP) system, the crop monitoring system, under controlled environmental conditions. The experiment was conducted over a period of 5 days, incorporating a water treatment regime having optimal irrigation and induced drought stress. Irrigation treatments represented either full irrigation (780 mL or restricted irrigation (260 mL). The study experiment was laid out in a randomized design with three replicates for each treatment. To validate the HTP dataset, several measurement techniques were employed: hyperspectral reflectance (daytime), RGB imaging (daytime), and chlorophyll fluorescence (nighttime), were conducted to assess plant health and stress responses. Soil moisture level was continuously monitored synchronously with each scan. Additionally, phenotypic traits were also measured using an automated multispectral imager (e.g., TraitFinder, Phenospex, Heerlen, the Netherlands) to validate the crop monitoring system.

### Trait Measurements.

Canopy hyperspectral reflectance was measured by the spectrometer. This instrument measures reflectance with a wavelength range of 500-900 nm. The field of view (FOV) of the bare fiber-optic probe was 25° (see FIG. 10). The spectrum of a white reference panel with known reflectance properties was acquired to derive the reflectance of the target. Measurements were acquired on canopies of targeted plants, distributed randomize manner under the crop monitoring system. The vegetation spectrum was measured from a distance of 75 cm above the plant canopies, with a spot size of approximately 16.80 cm². Then, statistical analyses were performed. The reflectance measurements were averaged over 10 nm to reduce collinearity and overfitting. In this way, 5 derived reflectance variables were calculated; the name of the variables indicated the central wavelength. The spectral indices used in this study are reported in Table 1.

Table 1. Vegetation indices (VIs) derived from hyperspectral reflectance. Rn indicates the reflectance value at wavelength n. All equations were calculated using values averaged across ten pixels centered at the wavelength of interest. *The traditional NDVI equation is presented here for user reference. For this study, a modified NDVI was calculated from PhenoGazer using a slightly lower far-red wavelength because growth chamber lights did not include the near-infrared wavelength used in traditional NDVI. The Instrument column indicates the system from which each VI was derived, with PG representing PhenoGazer and TF representing TraitFinder. Equations were obtained from the Index DataBase, https://www.indexdatabase.de/.

TABLE 1

| Acronym | Name | Equation | Instrument |
|---|---|---|---|
| NDVI | Normalized Difference Vegetation Index* | (R₈₀₀ − R₆₇₀)/(R₈₀₀ + R₆₇₀) | TF |
| mNDVI | Modified Normalized Difference | (R₇₇₅ − R₆₇₀)/(R₇₇₅ + R₆₇₀) | PG |
| | Vegetation Index | | |
| CIgreen | Green Chlorophyll Index | (R₇₃₀/R₅₃₀) − 1 | PG |
| PRI | Photochemical Reflectance Index | (R₅₃₁ − R₅₇₀)/(R₅₃₁ + R₅₇₀) | PG |
| CIrededge710 | Chlorophyll Red-Edge | (R₇₅₀/R₇₁₀) − 1 | PG |
| PSRI | Plant Senescence Reflectance Index | (R₆₇₈ − R₅₂₅)/R₇₅₀ | PG, TF |
| GLI | Green Leaf Index | (2*Green − Red − Blue)/(2* | TF |
| | | Green + Red + Blue); Blue = | |
| | | 420-480 nm; Green = 490- | |
| | | 570 nm; Red = 640-760 nm | |

Nighttime blue LED light induced chlorophyll fluorescence was also measured by integrating blue LED lights with spectrometer fibers over the same targeted plant canopies scanned for daytime reflectance. Vegetation indices such as NDVI were also measured using TraitFinder based multispectral imaging to validate the Vis derived from the crop monitoring system.

### Controlled Environment Conditions.

The experiment was carried out in a growth chamber configured to simulate natural environmental conditions with controlled variations: A 14-hour photoperiod was maintained from 8:00 AM to 10:00 PM. Light intensity was set at 600 photosynthetic photon flux density (PPFD) during the 1-hour periods at the start and end of the light cycle and increased to 1200 PPFD during the midday peak (3:00 PM). Day and night temperatures were maintained at 28° C. and 20° C., respectively. Relative humidity was consistently held at 50%. The CO2 concentration within the chamber was maintained at 450 ppm to facilitate normal photosynthetic activity. Hourly measurements were taken to monitor the response of the plants to the different water treatments during daytime. These measurements included both daytime and nighttime data collection, ensuring comprehensive monitoring of plant physiological responses throughout the diurnal cycle.

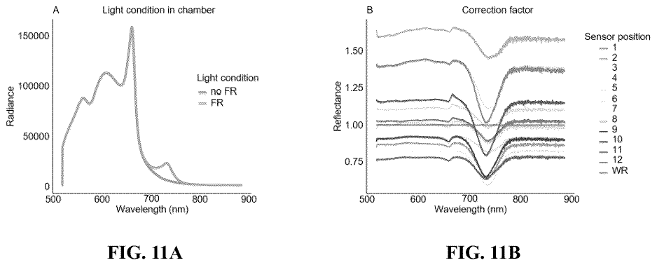

FIG. 11A illustrates a graph of a light condition in the chamber and FIG. 11B illustrates a graph of correction factors of the system for monitoring crops of the present patent document. FIG. 11A shows Lighting Conditions in the Growth Chamber which displays the LED radiance spectrum, illustrating the spectral output of combined white and far-red (FR) LEDs. FIG. 11 B presents reflectance correction factors applied for calibrating sensor-measured reflectance across different positions within the chamber.

### Results

Chamber light condition evaluation. To ensure comparable spectral responses across HTP measurements, it is important to account for environmental variability across the measurements. This is critical in field and greenhouse settings, but even present (albeit at lesser degrees of variation) in controlled environment chambers. During system testing, the experiment observed inconsistencies in light emitted from the LEDs distributed in the walk-in growth chamber (see FIG. 11). Such light inconsistencies will vary in all sorts of environments where overhead HTP systems are deployed due to differing combinations of shadows, reflections and light angles. As a result, a chamber calibration routine was developed to generate correction factors to account for shadow and chamber LED discrepancies across different channel positions within the chamber, which can be easily adapted to any deployment situation.

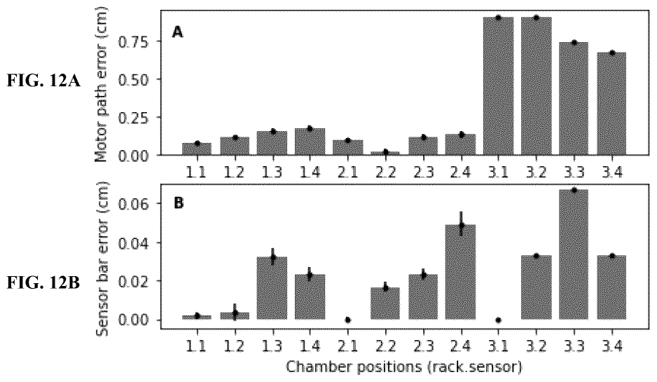

FIG. 12A illustrates a graph of motor path error positions versus chamber positions and FIG. 12B illustrates a graph of sensor bar error positions versus chamber positions of the system for monitoring crops of the present patent document.

In FIG. 12A, positional reliability of the crop monitoring system is shown, where FIG. 12 A shows positional error along the motor path, i.e. rack movement axis, indicating positional stopping accuracy by the HE sensors. In FIG. 12 B, positional error along the sensor bar is shown, indicating effects of any movement vibration on image and spectral acquisition. In FIG. 12A and FIG. 12B, the X-axis labels represent images taken at each unique chamber position (rack position followed by sensor position). The bars represent mean±SE of n=36 images per chamber position.

### System Reliability Test.

The experiment evaluated the reliability of the crop monitoring system movement and spectral acquisition to ensure robust system performance. Specifically, the experiment evaluated the repeatability of position by the movement module, by utilizing images to identify positional error (FIG. 12A and FIG. 12B). These figures illustrate performance of good and bad HE sensors. The experiment found higher positional error occurring at rack position 3 (FIG. 12A) due to a malfunctioning HE sensor, which triggered the system to return home and retry reaching the target position. Response at good sensors, located at P1 and P2, showed very little motor path error (<0.25 cm on the ground). Furthermore, the sensor bars exhibited very low influence on positional accuracy of image acquisition (FIG. 12B). This axis of error would be caused by residual vibration on the sensor bar after motor movement. Together, these results demonstrate the precision of the crop monitoring system which is critical for resampling plant targets during automated long-term monitoring.

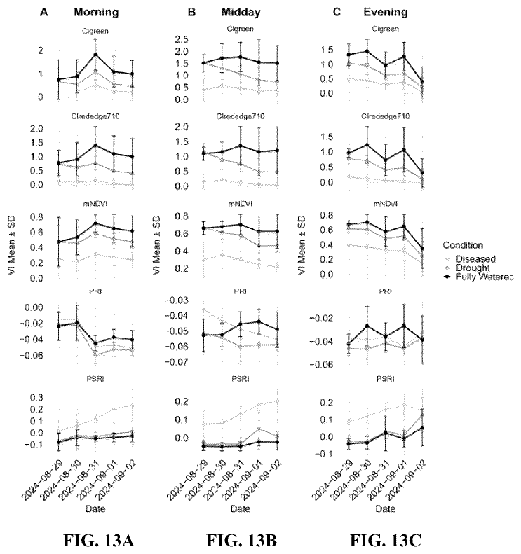

FIGS. 13A, 13B, and 13C illustrate graphs of conditions of the target plants during an experiment. FIGS. 13A, 13B, and 13C show diurnal and temporal variations in vegetation for diseased, drought and fully watered plants. In FIGS. 13A, 13B, and 13C temporal variations in vegetation indices for diseased, drought and fully watered plants are shown, measured during A) morning (1.5 h after lights on, at 600 PPFD), B) midday (1.5 h after shift to 1200 PPFD), and C) evening (0.5 h before lights off, at 600 PPFD). Error bars indicate mean+/−SD, n=4.

### Application Demonstration.

Variation in vegetation indices under varying environments. Here a series of vegetation indices (VIs) were calculated (Table 1). Note that because the artificial LEDs in the walk-in chamber used in this study support only 400-750 nm range, a modified Normalized Difference Vegetation Index (NDVI) (mNDVI) was calculated using 750 instead of 800 nm. FIG. 13 shows depicting the values of five different vegetation indices such as CIrededge710, CIgreen, mNDVI, and PRI measured across multiple times and dates for plants under three different conditions (Fully Watered, Drought, Diseased). FIGS. 13A, 13B, and 13C represent measurements taken at three different times of the day (morning, midday, and evening), allowing us to assess the diurnal variability of these indices under varying environmental stresses. The indices might vary in their diurnal patterns, reflecting the physiological responses of plants to the environmental conditions throughout the day. Note that differences across measurements on the same day can also indicate responses of the plants to leaf angles due to changing light intensity, as soybean plants are strongly heliotropic. Indeed, the results demonstrated that midday measurements, taken under high light conditions (1200 PPFD), showed the clearest treatment effects, as morning and evening measurements contained an additional factor of leaf angle changes in response to low light intensity (600 PPFD). Across the days of the experiment, stressed plants differed in response of most VIs compared to fully watered healthy plants.

CIrededge710, CIgreen and mNDVI are all VIs that reflect canopy greenness [27-29], and highly similar responses in these parameters were observed across treatments. The well-watered treatment exhibited consistently high values for CIrededge710, CIgreen and mNDVI over the experiment, indicative of healthy vegetation status with more green biomass. The droughted treatment initially exhibited similar values for all VIs to the well-watered treatment (all were healthy plants at the start of the experiment), but consistently deviated thereafter, with decreasing values of CIrededge710, CIgreen and mNDVI, reflecting the advancement of stress reducing canopy greenness and overall plant health. The diseased treatment, which was already heavily affected by disease at the start of the experiment, exhibited consistently low values of CIrededge710, CIgreen and NDVI, which continued to decline through the end of the experiment.

PRI is a VI that reflects the xanthophyll activation state as well as photochemical activity and is a good indicator of changes in photosynthetic efficiency and light use efficiency, with negative or reduced values indicating a decline in photosynthetic activity under stress [30,31]). The response of PRI among treatments was similar to that of the chlorophyll-based indices CIrededge710, CIgreen and mNDVI. The study observed consistently higher PRI from well-watered healthy plants, while PRI continuously decreased in both droughted and diseased plants throughout the experiment at all time points, with the lowest response from diseased plants.

PSRI reflects plant senescence and respond inversely from the greenness-based VIs above. Consistently high PSRI values were estimated under both stress conditions compared to fully watered plants, with stable high PSRI values in diseased plants and increasing PSRI values in droughted plants during the experiment at all times of the day.

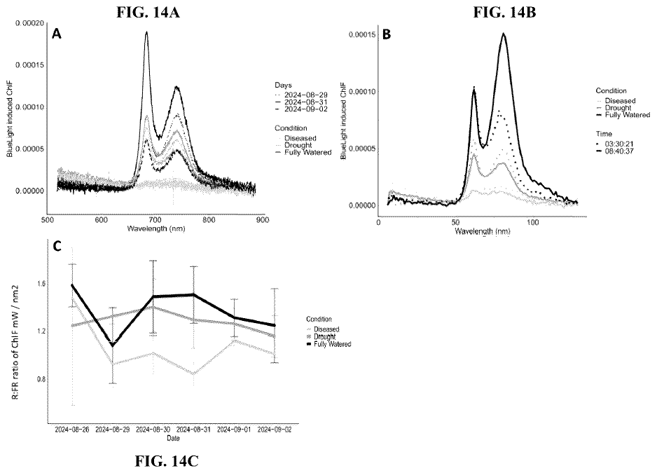

FIGS. 14A and 14B illustrate graphs of blue light induced chlorophyll fluorescence patterns, and FIG. 14C illustrates R:FR Ratio in various conditions during an experiment. A plant's R:FR ratio refers to the ratio of red light to far-red light that it receives. FIGS. 14A, 14B, and 14C show blue light induced chlorophyll fluorescence patterns and R:FR Ratio over various plant conditions. FIG. 14A shows dynamic fluorescence patterns across different wavelengths (500-900 nm), displaying responses from plants under three conditions: diseased, drought, and fully watered, over selected dates. FIG. 14B shows representative fluorescence patterns over two time points of the same night, from the third night of drought treatment. FIG. 14C shows the ratio of red to far-red chlorophyll fluorescence emission trends over time across the three conditions.

Nighttime blue light-induced chlorophyll fluorescence detects plant stress response. The experiment monitored chlorophyll fluorescence at different times during the night to assess whether any changes in the canopy occurred from 1 h after lights out compared with pre-dawn, and over drought treatment. Response of nighttime chlorophyll fluorescence are shown in FIG. 14.

FIG. 14 demonstrates the impact of water and disease stress on plant physiological processes, evident through alterations in chlorophyll fluorescence and R:FR ratios. Drought and disease stress notably diminishes chlorophyll fluorescence and alters the R:FR ratio, reflecting changes in photosynthetic activity and light absorption (FIGS. 14A and 14C). These results underscored the sensitivity of the photosynthetic apparatus to drought and its adaptive response to fluctuating water conditions. The fluorescence spectra demonstrate significant variations, with peaks around 685 nm (red) and 735 nm (far-red) indicating photosynthetic activity. Drought and disease decreased chlorophyll fluorescence emission, indicating reduced photosynthetic efficiency compared to plants that are fully watered (FIG. 14A,B). FIG. 14B shows a detailed view of how fluorescence changes within a brief timeframe of midnight to early in the morning. There are clear differences in the intensity of the fluorescence between stressed and fully watered conditions. The fluorescence is consistently lower in drought-stressed plants, indicating an immediate impact of water stress on photosynthetic function. The R:FR ratio is a critical indicator of how plants utilize light, particularly in response to their environment. Under disease conditions, the R:FR ratio is reduced, suggesting an adaptation or stress response affecting how red and far-red light is processed. As shown in FIG. 14C, variability in this ratio in healthy versus diseased plants underscores the dynamic nature of plant responses to stress.

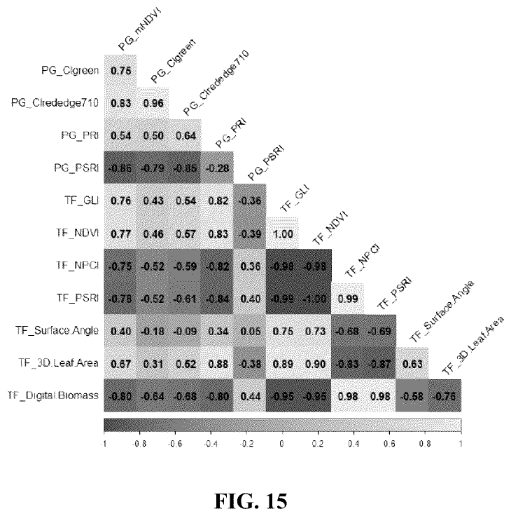

FIG. 15 illustrates graphs of Pearson correlations between the crop monitoring system based VIs and Phenospex parameters during an experiment. FIG. 15 shows Pearson correlations among vegetative indices and traits measured by the crop monitoring system (PhenoGazer, PG) and a commercial system (TraitFinder, TF). Low and high r values is presented by light to dark blue color circles while negative correlations between the traits is shown by light to dark maroon color. Abbreviations: PG, PhenoGazer; TF, TraitFinder.

### Correlation Between the Crop Monitoring System and TraitFinder Based Traits.

Some key metrics captured by the TraitFinder, a commercially available multispectral imaging plant phenotyping system were measured over the drought and fully watered plant to validate the crop monitoring system-based VI measurements. Correlations are shown in FIG. 15. Strong correlations (r=0.77) were observed between the crop monitoring system-based mNDVI and TraitFinder-based NDVI, as well as strong correlation between the crop monitoring system-based PRI and TraitFinder-based GLI, NDVI, and 3D leaf area (r>0.80). In contrast, negative correlations were found between the crop monitoring system-based PRI and Traitfinder-based digital biomass, NPCI and PSRI (r<−0.80). Crop monitoring system-based CIgreen, CIrededge710, and PSRI exhibited weaker correlations with TraitFinder-based measurements.

### Discussion

### Accounting for Variability on Light Environment.

The observed discrepancies in light conditions within controlled environment chambers necessitate a comprehensive understanding and correction strategy to ensure uniformity in experimental results. This study identifies substantial variability in the spectral quality of light, primarily due to the spatial arrangement and interaction of LED light sources in the chamber. FIG. 11A clearly shows the spectral differences when far-red (FR) light is included versus when it is absent, indicating how different light treatments can significantly alter the light environment within the chamber. Such variations can have profound effects on plant growth and physiological responses, which are often sensitive to narrow spectral changes. Therefore, the development of a calibration routine to correct for these inconsistencies, as outlined in the manuscript, is crucial for enhancing the reliability of high-throughput phenotyping systems.

Further analysis, as illustrated in FIG. 11B, reveals the impact of sensor position on light detection, with certain positions experiencing more pronounced disparities in light intensity and spectral composition. The correction factors developed range from minor adjustments to significant alterations, over 1.5 times the unadjusted readings, emphasizing the non-uniform distribution of light within the chamber. This underpins the importance of a dynamic correction routine that can be adapted to various chamber configurations and experimental setups. By implementing such corrective measures, researchers can mitigate the effects of light variability and ensure that differences in experimental outcomes are due to biological factors rather than environmental inconsistencies.

Lastly, the integration of these correction factors into standard experimental protocols promises to improve the accuracy and reproducibility of measurements across different studies and installations. The physical setup of LEDs and sensors can greatly influence the measurement and interpretation of light data. The ability to standardize these conditions, irrespective of the chamber design or the specific requirements of different research projects, is a significant advancement in the field of plant sciences. By addressing the challenges of light variability, this approach sets a new standard for controlled environment research, enabling more precise control over experimental variables and better comparability across studies.

Precision Positioning Confirmed with Computer Vision.

The crop monitoring system, designed for high throughput phenotyping, emphasizes the critical importance of precision in positioning over plant canopies to ensure accurate data acquisition. As indicated by the results presented in FIG. 12, the system exhibits remarkable precision in positional accuracy. Specifically, the motor path errors are predominantly less than 0.25 cm (FIG. 12A), showcasing the system's capability to maintain strict adherence to predetermined pathways, crucial for consistent spectral and image data collection. Such precision is vital as even minor deviations can lead to significant discrepancies in targeted plant canopies and subsequent physiological assessments of plant canopies.

In the case of sensor bar errors, the findings highlight the system's ability to mitigate the effects of residual vibration and other mechanical influences, maintaining sensor accuracy with deviations as minimal as 0.02 cm (FIG. 12B). This level of accuracy is particularly significant in a phenotyping context where high-resolution spatial data is essential for accurate trait assessment. The low error rates across different rack positions and sensors demonstrate the robustness of the system's design and its suitability for long-term monitoring tasks where precision is paramount to the reliability of the data collected.

Precision in positioning of the crop monitoring system enhances its capability to perform, thereby ensuring the accuracy of plant phenotyping efforts. This precision is crucial for differentiating subtle phenotypic variations among plant canopies, which are often critical for genetic research and crop improvement programs. The system's ability to maintain high positional accuracy over plant canopies ensures that each plant is monitored under consistent environmental conditions, thus minimizing data variability and enhancing the interpretability of phenotypic responses. This leads to more reliable data, facilitating precise assessments of plant characteristics that are essential for effective breeding and cultivation practices.

### System Sensitivity to Plant Stress Responses.

The sensitivity of the crop monitoring system in detecting plant stress responses is thoroughly demonstrated through both chlorophyll fluorescence under nighttime conditions and vegetation indices measurements. The results shown in FIG. 14 and FIG. 13 collectively highlight the system's capability to discern subtle physiological processes and environmental stress impacts on plants. Dynamic fluorescence patterns (FIG. 14A) and specific fluorescence profiles captured during brief timeframes (FIG. 14B) illustrate the system's precision in capturing real-time changes, with different fluorescence intensities reflecting the plant's immediate reaction to varying conditions of water and disease stress.

Vegetation indices such as CIrededge, CIGreen, mNDVI, and PRI, analyzed at different times of the day under various stress conditions (FIG. 13), provide complementary data that enhance the understanding of plant responses. The diurnal and temporal variations in these indices capture the physiological responses of plants to environmental conditions throughout the day, showcasing the indices' variability under stressed and fully watered conditions. Notably, indices like mNDVI exhibit significant sensitivity to stress, revealing reductions in greenness and density that correspond with environmental stressors such as drought and disease.

The integration of R-FR ratio trends from FIG. 14C further corroborates the sensitivity of the crop monitoring system to varying physiological states of plants. These ratios provide a direct measure of changes in photosynthetic efficiency and stress adaptation mechanisms, showing distinct patterns across fully watered, drought, and diseased conditions. This specificity in response detection allows researchers to track plant health and stress adaptation over time with high accuracy, crucial for understanding the mechanistic responses of plants to environmental stresses.

Together, the combination of advanced imaging for ChlF and the analytical depth provided by multiple vegetation indices ensure that the crop monitoring system remains a vital tool in the field of plant phenotyping. This capability is fundamental for advancing the understanding of plant physiology and enhancing the efficacy of breeding programs targeted at stress resistance.

The crop monitoring system demonstrates significant capabilities in monitoring plant stress responses. Several advancements may be made to further broaden the utility of the crop monitoring system. First, integrating adaptive algorithms that can compensate for environmental variability would enhance the robustness of data collection, making it more reliable under diverse conditions. Furthermore, expanding the range of usable wavelengths and incorporating multispectral or hyperspectral imaging capabilities would allow for a more comprehensive analysis of plant health and stress responses. Such enhancements would enable the crop monitoring system to operate effectively in a wider range of environments and provide more detailed insights into plant physiology, aiding in the development of stress-resilient plant varieties.

The crop monitoring system has proven to be an invaluable tool for precise monitoring of plant stress responses through its integration of nighttime chlorophyll fluorescence and vegetation indices. By providing detailed insights into the diurnal and temporal variations in plant physiology under various stress conditions, the system enables researchers to accurately track environmental impacts on plant health and accelerate identification of more resilient crop varieties.

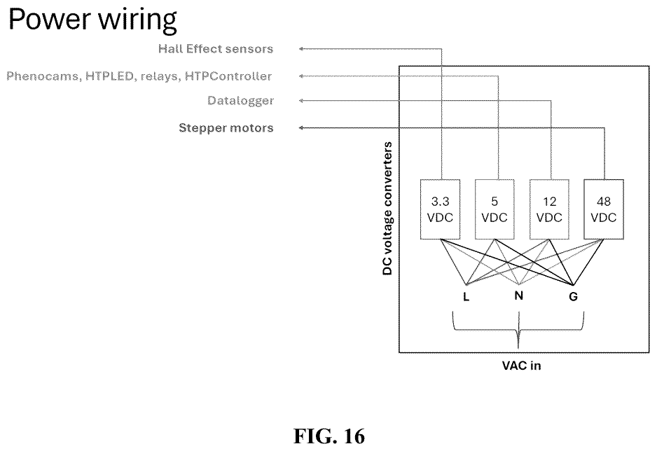

FIG. 16 illustrates a schematic diagram of a power wiring configuration of the system for monitoring crops of the present patent document. In the embodiment shown in FIG. 16, VDC refers to Volts Direct Current, and VAC refers to Volts Alternating Current.

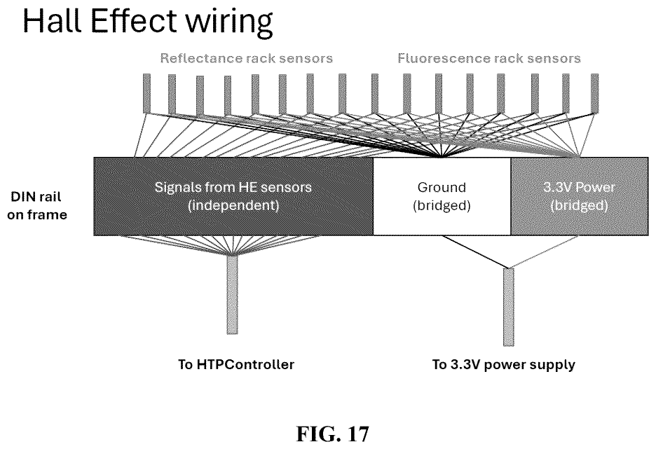

FIG. 17 illustrates a schematic diagram of a Hall effect wiring configuration of the system for monitoring crops of the present patent document. DIN rail mounted on the outer frame of the crop monitoring system. Blue block is used for data connections. White block is used for ground connections. Orange block is used for 3.3V power connections.

In some embodiments, instead of running each individual sensor directly to the HTPController, all HE sensors may be connected to a DIN rail mounted on the outer frame. The power and ground for the HE sensors may be supplied by an external 3.3V power supply so that they do not pose any risk to the HTPController.

Data signals inbound from the HE sensors are bundled into a DB-9 cable that feeds into the instrument enclosure and splits back out to GPIO connectors attached to the HTPController. Each HE sensor also now incorporates a 3.3K ohms external pull-up resistor instead of relying on the built-in 50-65K ohms internal pull-up resistors on the Raspberry Pi, to improve signal stability when sensors are not active. In some embodiments, the multiplexer and Kee board may be removed from the motor module.

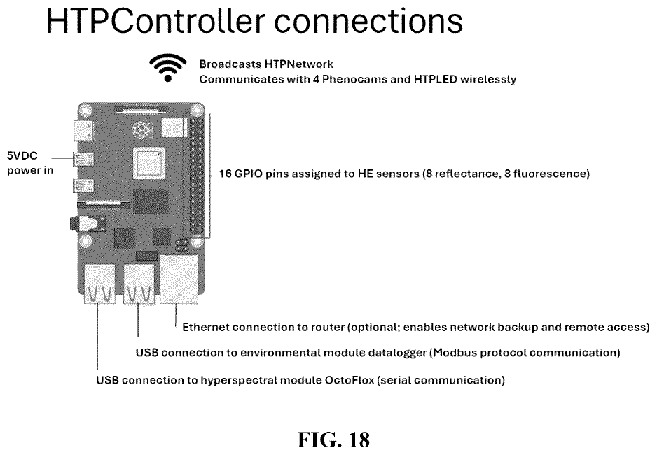

FIG. 18 illustrates a schematic diagram of a main controller of the system for monitoring crops of the present patent document. The main controller may have 16 GPIO pins assigned to HE sensors (8 reflectance, 8 fluorescence), an optional Ethernet connection to a router (enables network backup and remote access), a USB connection to an environmental module datalogger (e.g., Modbus protocol communication), and a USB connection to a hyperspectral module (e.g., OctoFlox) (serial communication). The main controller may broadcast to a network, and communicate with the image modules and light controller wirelessly.

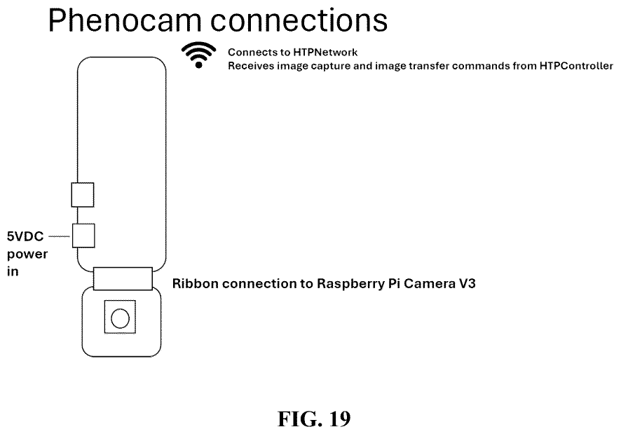

FIG. 19 illustrates a schematic diagram of an imaging module of the system for monitoring crops of the present patent document. The imaging module may be connected to a LAN, where the imaging module may receive image capture and image transfer commands from the main controller. The imaging controller may be connected to a camera by a ribbon connection.

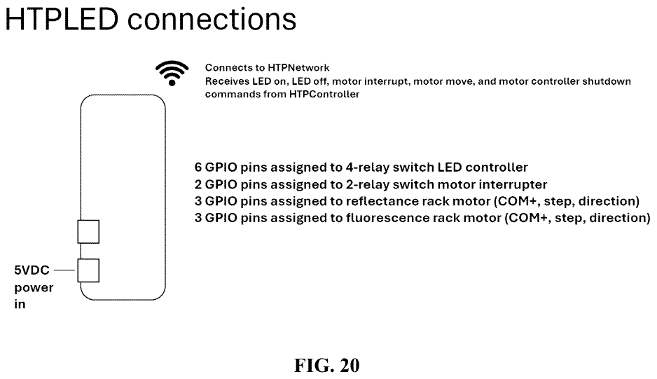

FIG. 20 illustrates a schematic diagram of a controller with LED connections of the system for monitoring crops of the present patent document. The lighting module may be connected to a LAN, where the lighting module may receive LED on, LED off, motor interrupt, motor move, and motor controller shutdown commands from the main controller. In the embodiment shown in FIG. 20, the lighting controller may have 6 General Purpose Input/Output (GPIO) pins assigned to 4-relay switch LED controller, 2 GPIO pins assigned to 2-relay switch motor interrupter, 3 GPIO pins assigned to reflectance rack motor (COM+, step, direction), and 3 GPIO pins assigned to fluorescence rack motor (COM+, step, direction).

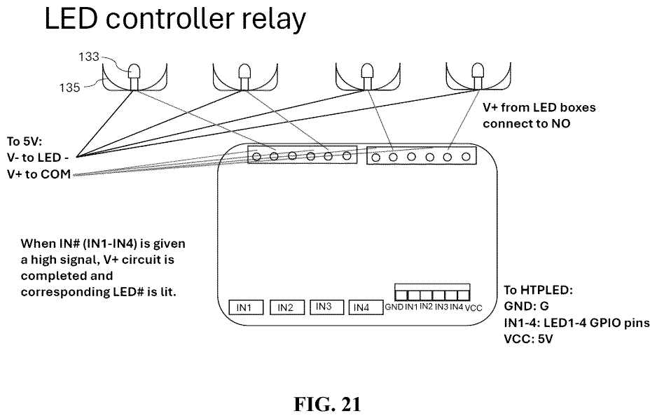

FIG. 21 illustrates a schematic diagram of an LED controller relay of the system for monitoring crops of the present patent document. In the embodiment shown in FIG. 21, when IN #(IN1-IN4) is given a high signal, the V+ circuit is completed and the corresponding LED number is turned on. In the embodiment shown in FIG. 21, GND refers to an electrical ground, and VCC refers to a voltage common collector.

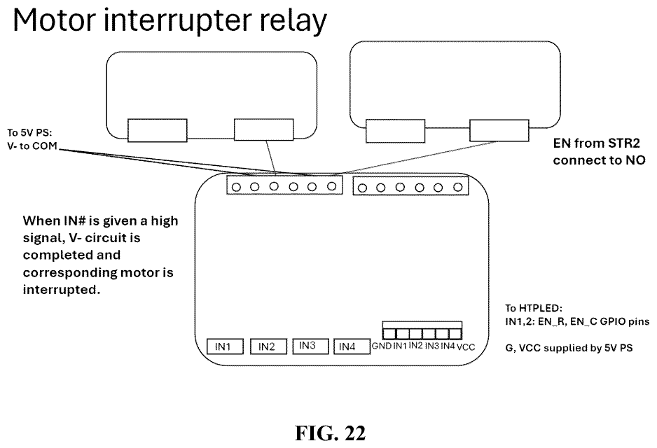

FIG. 22 illustrates a schematic diagram of a motor interrupt relay of the system for monitoring crops of the present patent document. In the embodiment shown in FIG. 22, when IN #(IN1-IN4) is given a high signal, the V-circuit is completed and the corresponding motor is interrupted.

Although the embodiments have been described with reference to the drawings and specific examples, it will readily be appreciated by those skilled in the art that many modifications and adaptations of the apparatus and processes described herein are possible without departure from the spirit and scope of the embodiments as claimed hereinafter. Thus, it is to be clearly understood that this description is made only by way of example and not as a limitation on the scope of the embodiments as claimed below.

For the foregoing reasons, the subject matter described herein provides innovative systems and methods for monitoring crops. The current system may be modified in multiple ways and applied in various technological applications. The disclosed apparatus, systems, and methods may be modified and customized as required by a specific operation or application, and the individual components may be modified and defined, as required, to achieve the desired result.

Although the materials of construction may not be described, they may include a variety of compositions consistent with the function described herein. Such variations are not to be regarded as a departure from the spirit and scope of this disclosure, and all such modifications as would be obvious to one skilled in the art are intended to be included within the scope of the following claims.

The technology is operational with numerous other general purpose or special purpose computing system environments or configurations. Examples of well-known computing systems, environments, and/or configurations that may be suitable for use with the development include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like. As used herein, the terms system, server, and module can refer to software applications, computers, network devices, databases, or other hardware or software capable of performing the described function. Servers, systems, and modules may be embodied on standalone computers, networked computers, or may be embodied on other portions of the system. Description of a component as a server, module, or a system does not limit the component thereto.

As used herein, instructions refer to computer-implemented steps for processing information in the system. Instructions can be implemented in software, firmware or hardware and include any type of programmed step undertaken by components of the system.

A microprocessor may be any conventional general purpose single- or multi-chip microprocessor such as a Pentium® processor, a Pentium® Pro processor, a 8051 processor, a MIPS® processor, a Power PC® processor, or an Alpha® processor. In addition, the microprocessor may be any conventional special purpose microprocessor such as a digital signal processor or a graphics processor. The microprocessor typically has conventional address lines, conventional data lines, and one or more conventional control lines.

The system may be used in connection with various operating systems such as Linux®, UNIX® or Microsoft Windows®. The system control may be written in any conventional programming language such as C, C++, BASIC, Pascal, or Java, and ran under a conventional operating system. C, C++, BASIC, Pascal, Java, and FORTRAN are industry standard programming languages for which many commercial compilers can be used to create executable code. The system control may also be written using interpreted languages such as Perl, Python or Ruby.

Those of skill will further recognize that the various illustrative logical blocks, modules, circuits, and algorithm steps described in connection with the embodiments disclosed herein may be implemented as electronic hardware, software stored on a computer readable medium and executable by a processor, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the present development.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

If implemented in software, the functions may be stored on or transmitted over as one or more instructions or code on a computer-readable medium. The steps of a method or algorithm disclosed herein may be implemented in a processor-executable software module which may reside on a computer-readable medium. Computer-readable media includes both computer storage media and communication media including any medium that can be enabled to transfer a computer program from one place to another. A storage media may be any available media that may be accessed by a computer. By way of example, and not limitation, such computer-readable media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

The amounts, percentages and ranges disclosed in this specification are not meant to be limiting, and increments between the recited amounts, percentages and ranges are specifically envisioned as part of the invention. All ranges and parameters disclosed herein are understood to encompass any and all sub-ranges subsumed therein, and every number between the endpoints. For example, a stated range of “1 to 10” should be considered to include any and all sub-ranges between (and inclusive of) the minimum value of 1 and the maximum value of 10 including all integer values and decimal values; that is, all sub-ranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions, and so forth as used in the specification and claims are to be understood as being modified in all instances by the implied term “about.” The (stated or implied) term “about” indicates that a numerically quantifiable measurement is assumed to vary by as much as 30 percent, but preferably by at least 10%. Essentially, as used herein, the term “about” refers to a quantity, level, value, or amount that varies by as much 10% to a reference quantity, level, value, or amount. Accordingly, unless otherwise indicated, the numerical properties set forth in the following specification and claims are approximations that may vary depending on the desired properties sought to be obtained in embodiments of the present invention.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein.

The foregoing description details certain embodiments of the systems, devices, and methods disclosed herein. It will be appreciated, however, that no matter how detailed the foregoing appears in text, the systems, devices, and methods can be practiced in many ways. It should be noted that the use of particular terminology when describing certain features or aspects of the development should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element which is not specifically disclosed herein. The term “an effective amount” as applied to a component or a function excludes trace amounts of the component, or the presence of a component or a function in a form or a way that one of ordinary skill would consider not to have a material effect on an associated product or process.

What is claimed is:

1. A crop monitoring system, the system comprising:
a first support frame, the support frame comprising:
a first sensor rack coupled to the first support frame, the first sensor rack comprising a first sensor module;
a movement module, the movement module comprising a first sensor rack motor, wherein the first sensor rack motor is configured to move the first sensor rack along the first support frame from a first starting location to a first ending location; and
a hyperspectral module, wherein the first sensor module is coupled to the hyperspectral module.

2. The system of claim 1, further comprising:
a second sensor rack coupled to a second support frame, the second sensor rack comprising a second sensor module.

3. The system of claim 2, wherein the movement module further comprises a second sensor rack motor, the second sensor rack motor configured to move the second sensor rack along the second support frame from a second starting location to a second ending location.

4. The system of claim 3, further comprising:
a controller in electrical communication with the movement module, the controller configured to:
control the movement of the first sensor rack and the second sensor rack.

5. The system of claim 4, further comprising:
an environmental module, the environmental module configured to collect a light intensity; and
wherein the controller is in electrical communication with the environmental module to determine a daytime cycle lighting condition or a nighttime cycle lighting condition based on the light intensity from the environmental module.

6. The system of claim 5, wherein the crop monitoring system is an autonomous crop monitoring system.

7. The system of claim 1, wherein the first sensor module further comprises a light source, wherein the light source is configured to induce a chlorophyll fluorescence in a subject crop.

8. The system of claim 3, wherein the second sensor module further comprises an imaging module, wherein the imaging module comprises a camera, wherein the camera is configured to collect above-canopy imagery of a subject crop.

9. The system of claim 1, wherein the hyperspectral module comprises a spectrometer configured to a spectral range of about 500-900 nm.

10. The system of claim 6, further comprising:
a Hall effect sensor, where the Hall effect sensor is coupled to the first support frame.

11. The system of claim 10, further comprising:
a magnet coupled to the first sensor rack, wherein the Hall effect sensor is configured to sense the magnet and send a signal to the controller to stop the movement of the first sensor rack such that the first sensor rack is over a subject crop.

12. The system of claim 5, wherein the first sensor rack is located below the second sensor rack, such that the first sensor rack is configured to travel parallel to the second sensor rack between a subject crop and the second sensor rack.

13. The system of claim 9, wherein the first sensor module comprises a fiber optic sensor, and wherein the fiber optic sensor is coupled to the hyperspectral module.

14. The system of claim 6, further comprising:
a first drive shaft, wherein the first drive shaft is operatively connected to the first sensor rack motor;
a first drive chain operatively connected to the first drive shaft and a first front top axle; and
a first rack chain operatively connected to the first front top axle and a first front bottom axle at a front end of the first support frame, the first rack chain operatively connected to the first sensor rack.

15. An automated crop monitoring system, the system comprising:
a control module, configured to direct operations of the system;
a first support frame;
a first sensor rack coupled to the first support frame, the first sensor rack comprising at least one sensor coupled to the first sensor rack;
a movement module, the movement module comprising a first sensor rack motor, wherein the first sensor rack motor is configured to move the first sensor rack along the first support frame from a first starting location to a first ending location;
at least one Hall effect sensor coupled to first support frame; and
a magnet coupled to the first sensor rack, wherein the at least one Hall effect sensor is configured to sense the magnet and send a signal to the control module to stop the movement of the first sensor rack such that the first sensor rack is over a subject crop.

16. The system of claim 15, further comprising:
a hyperspectral module, wherein the at least one sensor is coupled to the hyperspectral module.

17. The system of claim 15, further comprising:
a second sensor rack coupled to a second support frame, the second sensor rack comprising a second sensor module.

18. The system of claim 17, wherein the movement module further comprises a second sensor rack motor, the second sensor rack motor configured to move the second sensor rack along the second support frame from a second starting location to a second ending location.

19. The system of claim 18, further comprising:
an environmental module, the environmental module configured to collect a light intensity; and
wherein the control module is in electrical communication with the environmental module to determine a daytime cycle lighting condition or a nighttime cycle lighting condition based on the light intensity from the environmental module.

20. The system of claim 19, further comprising a light module, wherein the light module comprises a light source, and wherein the light source is configured to induce a chlorophyll fluorescence in a subject crop.

21. The system of claim 20, wherein the second sensor module further comprises an imaging module, wherein the imaging module comprises a camera, wherein the camera is configured to collect above-canopy RGB imagery of the subject crop.

22. The system of claim 21, further comprising:
a first drive shaft, wherein the first drive shaft is operatively connected to the first sensor rack motor;
a first drive chain operatively connected to the first drive shaft and a first front top axle; and
a first rack chain operatively connected to the first front top axle and a first front bottom axle at a front end of the first support frame, the first rack chain operatively connected to the first sensor rack.

23. A method for monitoring crop performance, the method comprising the steps of:
determining a nighttime cycle;
moving and aligning a first sensor rack to a configuration to sense and record data from a subject crop during the nighttime cycle;
determining a daytime cycle; and
moving and aligning a second sensor rack to a configuration to sense and record data from the subject crop during the daytime cycle.

24. The method of claim 23, further comprising the step of:
scanning a white reference at a home position of the second sensor rack after determining the daytime cycle.

25. The method of claim 24, further comprising the steps of:
capturing an image at a first sensor position and transferring the image to a main controller; and
taking a measurement at the first sensor position by a hyperspectral sensor.

26. The method of claim 25, further comprising the steps of:
calculating a radiance using a radiometric calibration file;
calculating a raw reflectance;
calculating a corrected reflectance;
saving at least one of a raw spectral data, a radiance, a raw reflectance, and a corrected reflectance;
moving the second sensor rack to a home position; and
taking at least one environmental measurement.

27. The method of claim 23, further comprising the steps of:
turning on a blue light at a first sensor position;
taking an image of a subject crop at the first sensor position;
transferring the image to the main controller; and
taking a sample measurement at the first sensor position.

28. The method of claim 27, further comprising the steps of:
calculating a fluorescence using a radiometric calibration file;
saving at least one of a raw spectral data and a fluorescence;
moving the first sensor rack to a home position; and
taking at least one environmental measurement.
